(19) United States
(12) Patent Application Publication
(10) Pub. No.: US 2026/0226148 A1
(43) Pub. Date: Aug. 6, 2026

(54) PROTECTION OF THERAPEUTIC PROTEINS

(71) Applicant: AMGEN INC., Thousand Oaks, CA (US)

(72) Inventors: Kyle Shamus MCELEARNEY, Newbury Park, CA (US); Quanzhou LUO, Oak Park, CA (US); Shivani GUPTA, Foster City, CA (US); Rinaben Bhavin GANDHI, Cambridge, MA (US); Sneha SUMAN, Watertown, MA (US); Bhavana SHAH, Thousand Oaks, CA (US); Jennitte LeAnn STEVENS, Thousand Oaks, CA (US)

(21) Appl. No.: 18/708,152

(22) PCT Filed: Nov. 8, 2022
(86) PCT No.: PCT/US2022/079488

§ 371 Date: May 7, 2024

Related U.S. Application Data
(60) Provisional application No. 63/277,501, filed on Nov. 9, 2021.
(60) Provisional application No. 63/326,194, filed on Mar. 31, 2022.

(51) Int. Cl. C07K 16/24

(52) U.S. Cl. CPC C07K 16/244; C07K 2317/14; C07K 2317/21; C07K 2317/41

(57) ABSTRACT

The disclosure relates to methods of improving sialic acid content of a therapeutic protein comprising expressing an α2,6-sialyltransferase-1 (ST6) or both ST6 and β1,4 galactosyltransferase 1 (B4GALT1) in a CHO cell; and culturing the cell in a medium comprising galactose and manganese.

### RELATED APPLICATIONS

This application claims the benefit of U.S. Provisional Application No. 63/277,501, filed on Nov. 9, 2021, and Application No. 63/326,194, filed on Mar. 31, 2022, both of which are hereby incorporated by reference in their entireties.

### SEQUENCE LISTING

The present application is being filed along with a Sequence Listing in electronic format. The Sequence Listing is provided as a file entitled A-2752-WO01-SEC_Final_SeqListing_10252022, created Oct. 25, 2022, which is 49 KB in size. The information in the electronic format of the Sequence Listing is incorporated herein by reference in its entirety.

### FIELD OF THE INVENTION

The disclosure relates to methods of improving sialic acid content of a therapeutic protein comprising expressing an α2,6-sialyltransferase-1 (ST6) or both ST6 and β1,4 galactosyltransferase 1 (B4GALT1) in a CHO cell; and culturing the cell in a medium comprising galactose and manganese.

### BACKGROUND

Pharmaceutical biosynthesis of therapeutic proteins is complicated by the need for both high levels of expression and appropriate posttranslational processing, which involves the addition of N-linked and O-linked branched oligosaccharide chains.

In glycoproteins, sugars are attached either to the amide nitrogen atom in the side chain of asparagine (termed an N-linkage) or to the oxygen atom in the side chain of serine or threonine (termed an O-linkage). The process for forming N-linked carbohydrates begins with the addition of 14 monosaccharides to a lipid-linked dichol in the endoplasmic reticulum (ER). After its formation, this carbohydrate complex is then transferred to the protein by the oligosaccharyltransferase (OST) complex in a process termed “core glycosylation” in the ER. The oligosaccharyltransferase (OST) complex is a multi-protein unit comprised of ribophorin I, II, OST48 and DAD1 (Kelleher and Gilmore 1997 PNAS 94(10):4994-4999; Kelleher et al. 2003 Molecular Cell 12(1):101-111; and Kelleher et al. 1992 Cell 69(1):55-65).

Subsequently, the polypeptides are transported to the Golgi complex, where the 0-linked sugar chains are added, and the N-linked sugar chains are modified in many different ways. In the cis and medial compartments of the Golgi complex, the original 14-saccharide N-linked complex may be trimmed through removal of mannose (Man) residues and elongated through addition of N-acetylglucosamine (GlcNac) and/or fucose (Fuc) residues. The various forms of N-linked carbohydrates have in common a pentasaccharide core consisting of three mannose and two N-acetylglucosamine residues. Finally, in the trans Golgi, other GlcNac residues can be added, followed by galactose (Gal) and a terminal sialic acid (Sial). Carbohydrate processing in the Golgi complex is called “terminal glycosylation” to distinguish it from core glycosylation.

Sialic acid is a generic name for a family of about 30 naturally occurring acidic monosaccharides that are frequently the terminal sugars of carbohydrates found on glycoproteins and glycolipids. Sialylation of recombinant glycoproteins is very important and may impart many significant properties to the glycoprotein including charge, immunogenicity, resistance to protease degradation, plasma clearance rate, and bioactivity.

### SUMMARY

The present application is based on the discovery that (a) expressing an α2,6-sialyltransferase 1 (ST6) (SEQ ID NO: 1, 3, 5, 7, 9, or 11); or co-expressing a ST6 (SEQ ID NO: 1, 3, 5, 7, 9, or 11) and a β1,4 galactosyltransferase 1 (B4GALT1) (SEQ ID NO: 13, 15, 17, 19, 21, 23, 25, or 27) in the Chinese Hamster Ovary (CHO) cells and; optionally (b) culturing the cells in a medium comprising galactose and manganese can increase the sialic acid content in a recombinant antibody produced by said CHO cells (in particular, an increase in the N-Acetylneuraminic Acid (NANA) form of sialic acid with α2,6 linkage).

Accordingly, in one aspect, the invention provides a method of increasing sialic acid content of a therapeutic protein produced by a CHO cell comprising expressing an α2,6-sialyltransferase-1 (ST6) (SEQ ID NO: 1, 3, 5, 7, 9, or 11) in the CHO cell.

In another aspect, the invention provides a method of increasing sialic acid content of a therapeutic protein produced by a CHO cell comprising co-expressing an α2,6-sialyltransferase-1 (ST6) (SEQ ID NO: 1, 3, 5, 7, 9, or 11) and a β1,4-galactosyltransferase 1 (B4GALT1) (SEQ ID NO: 13, 15, 17, 19, 21, 23, 25, or 27) in the CHO cell.

In another aspect, provided is a method of increasing sialic acid content of a therapeutic protein produced by a CHO cell comprising: (a) expressing an α2,6-sialyltransferase 1 (ST6) (SEQ ID NO: 1, 3, 5, 7, 9, or 11) in the CHO cell; and (b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

In another aspect, provided is a method of increasing sialic acid content of a therapeutic protein produced by a CHO cell comprising: (a) expressing an α2,6-sialyltransferase-1 (ST6) (SEQ ID NO: 1, 3, 5, 7, 9, or 11) and a β1,4-galactosyltransferase 1 (B4GALT1) (SEQ ID NO: 13, 15, 17, 19, 21, 23, 25, or 27) in the CHO cell; and (b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

In some embodiments, the sialylation of the protein produced by the CHO cell is increased by at least 10% or at least 20% compared to a protein produced by a CHO cell cultured in a medium that does not comprise added manganese and galactose during the cell culture.

In some embodiments, the methods described herein comprise adding manganese and galactose to the medium on day 3 of the cell culture. In some embodiments, at least 100 ppb (Parts Per Billion) manganese and at least 15 mM galactose is added to the medium on day 3.

In some embodiments, the method described herein further comprises adding manganese and galactose to the medium on day 6. In some embodiments, at least 100 ppb manganese and at least 15 mM galactose is added to the medium on day 6.

In some embodiments, the method described herein further comprises adding manganese and galactose to the medium on day 8.

In some embodiments, the medium further comprises copper.

In some embodiments, the method described herein further comprises adding about 10 mM to about 100 mM galactose cumulatively over the culture period. In one example, the method comprises adding about 45 mM galactose cumulatively over the culture period.

In some embodiments, the method described herein further comprises adding about 40 ppb to about 400 ppb manganese cumulatively over the culture period. In one example, the method comprises adding about 400 ppb manganese cumulatively over the culture period.

In some embodiments, the method described herein further comprises adding about 0.01 mM to about 0.5 mM copper cumulatively over the culture period. In one example, the method comprises adding about 0.1 mM copper cumulatively over the culture period. In some embodiments, the therapeutic protein as described herein is a secreted and recombinant protein. In some embodiments, the therapeutic protein is an antibody or antigen-binding fragment thereof, a derivative of an antibody or antibody fragment, a bi-specific T-cell engager molecule, or a fusion polypeptide. In some embodiments, the antibody is an anti-IL12 antibody, an anti-IL23 antibody, or an anti-IL12/23 antibody (e.g., ustekinumab antibody comprising the heavy chain sequence according to SEQ ID NO: 29 and the light chain sequence according to SEQ ID NO: 30).

In some embodiments, the sialic acid described herein is α2,6-sialylated glycan. In some embodiments, the level of α2,6-sialylated glycan can be confirmed by hydrophilic interaction liquid chromatography (HILIC)-mass spectrometry (MS) analysis. In some embodiments, the level of α2,6-sialylated glycans can be kept constant in the protein produced with extended cell culture duration. In some embodiments, the extended cell culture duration is about 27 population doublings (PDL).

In some embodiments, the therapeutic protein (e.g., antibody) produced according to the method described herein can be used in treating plaque psoriasis, psoriatic arthritis, Crohn's disease, or ulcerative colitis.

### BRIEF DESCRIPTION OF THE FIGURES/DRAWINGS

FIG. 1A, FIG. 1B, FIG. 1C, FIG. 1D, and FIG. 1E show cell line stability for 3 clones (Clones 1-3) as assessed by evaluating changes in titer, mRNA expression of gene of interest, and glycan profile over defined duration and intervals of Population Doubling Levels (PDLs). Different glycans were evaluated using hydrophilic interaction liquid chromatography (HILIC)-mass spectrometry (MS) analysis for clone 1, 2 and 3 across OPDL, 30PDL and 50PDL at (A) % Sialic acid (B) % High Mannose (C) % Galactosylation (D) % Afucosylation, and (E) FITC-Sambucus nigra lectin (SNA1) staining for α-2,6 linkage. Each data point for a clone represent different PDL, and error bars show standard deviation between the duplicates.

### DETAILED DESCRIPTION

The present application is based on the discovery that (a) expressing an α2,6-sialyltransferase 1 (ST6) (SEQ ID NO: 1, 3, 5, 7, 9, or 11); or co-expressing a ST6 (SEQ ID NO: 1, 3, 5, 7, 9, or 11) and a β1,4 galactosyltransferase 1 (B4GALT1) (SEQ ID NO: 13, 15, 17, 19, 21, 23, 25, or 27) in the Chinese Hamster Ovary (CHO) cells and; optionally (b) culturing the cell in a medium comprising galactose and manganese can increase the sialic acid content in a recombinant antibody produced by said CHO cells (in particular, an increase in the N-Acetylneuraminic Acid (NANA) form of sialic acid with α-2, 6 linkage). Further, the addition of manganese and galactose to culture medium has been shown herein to result in significant alterations in post-translational processing of a therapeutic protein (e.g., an antigen binding protein, such as an antibody), by the cultured cells producing the therapeutic protein. As shown herein, the combination of manganese and galactose in the culture medium decreased the amount of lower sialylated glycoprotein produced and increased the amount of highly sialylated glycoprotein recovered.

Accordingly, in one aspect, the invention provides a method of increasing sialic acid content of a therapeutic protein produced by a CHO cell comprising expressing an α2,6-sialyltransferase-1 (ST6) (SEQ ID NO: 1, 3, 5, 7, 9, or 11) in the CHO cell.

In another aspect, the invention provides a method of increasing sialic acid content of a therapeutic protein produced by a CHO cell comprising co-expressing an α2,6-sialyltransferase-1 (ST6) (SEQ ID NO: 1, 3, 5, 7, 9, or 11) and a β1,4-galactosyltransferase 1 (B4GALT1) (SEQ ID NO: 13, 15, 17, 19, 21, 23, 25, or 27) in the CHO cell.

In another aspect, provided is a method of increasing sialic acid content of a therapeutic protein produced by a CHO cell comprising: (a) expressing an α2,6-sialyltransferase 1 (ST6) (SEQ ID NO: 1, 3, 5, 7, 9, or 11) in the CHO cell; and (b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

In another aspect, provided is a method of increasing sialic acid content of a therapeutic protein produced by a CHO cell comprising: (a) expressing an α2,6sialyltransferase1 (ST6) (SEQ ID NO: 1, 3, 5, 7, 9, or 11) and a β1,4galactosyltransferase 1 (B4GALT1) (SEQ ID NO: 13, 15, 17, 19, 21, 23, 25, or 27) in the CHO cell; and (b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

### Definitions

Sialylation, as used herein, is the addition of a sialic acid residue to a protein, which may be a glycoprotein.

The term sialic acid, as used herein, encompasses a family of sugars containing 9 or more carbon atoms, including a carboxyl group. A generic structure encompassing all known natural forms of sialic acid is shown below.

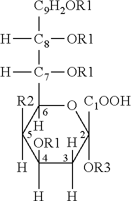

R1 groups at various positions on a single molecule can be the same as or different from each other. R1 can be a hydrogen or an acetyl, lactyl, methyl, sulfate, phosphate, anhydro, sialic acid, fucose, glucose, or galactose group. R2 can be an N-acetyl, N-glycolyl, amino, hydroxyl, N-glycolyl-O-acetyl, or N-glycolyl-O-methyl group. R3 represents the preceding sugar residue in an oligosaccharide to which sialic acid is attached in the context of a glycoprotein. R3 can be galactose (connected at its 3, 4, or 5 position), N-acetyl-galactose (connected at its 6 position), N-acetyl-glucose (connected at its 4 or 6 position), sialic acid (connected at its 8 or 9 position), or 5-N-glycolyl-neuraminic acid. Essentials of Glycobiology, Ch. 15, Varki et al., eds., Cold Spring Harbor Laboratory Press, New York (1999). More than 40 forms of sialic acid have been found in nature. Essentials of Glycobiology, Ch. 15, Varki et al., eds., Cold Spring Harbor Laboratory Press, New York (1999). A common form of sialic acid is N-acetylneuraminic acid (NANA), in which R1 is a hydrogen at all positions and R2 is an N-acetyl group.

The terms “operably linked” or “functionally linked” used herein refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation.

The term “promoter” refers to a nucleotide sequence, usually upstream (5′) to its coding sequence, which controls the expression of the coding sequence by providing the recognition site for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

A promoter may also contain DNA sequences that are involved in the binding of protein factors, which control the effectiveness of transcription initiation in response to physiological or developmental conditions. The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Techniques for the recombinant expression of enzymes in a cell and genetic modification of a mammalian cell (including CHO cells) are well known to those skilled in the art. Typically, such techniques involve transformation of a cell with nucleic acid construct comprising the relevant sequence. Such methods are, for example, known from standard handbooks, such as Sambrook and Russel (2001) “Molecular Cloning: A Laboratory Manual (3rd edition), Cold Spring Harbor Laboratory, Cold Spring Harbor Laboratory Press, or F. Ausubel et al., eds., “Current protocols in molecular biology”, Green Publishing and Wiley Interscience, New York (1987). Methods for transformation and genetic modification of fungal host cells are described in, e.g., European Application No. EP-A-0635574, International Patent Publication No. WO 98/46772, International Patent Publication No. WO 99/60102, International Patent Publication No. WO 00/37671, International Patent Publication No. WO 90/14423, European Application No. EP-A-0481008, European Application No. EP-A-0635574 and U.S. Pat. No. 6,265,186, the disclosures of which are incorporated herein by reference in their entireties.

As used herein, the term “antibody” refers to a protein having a conventional immunoglobulin format, comprising heavy and light chains, and comprising variable and constant regions. For example, an antibody can be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In exemplary aspects of the present disclosure, the therapeutic protein is an antigen binding fragment or an antibody. As used herein, the term “antigen binding antibody fragment” refers to a portion of an antibody that is capable of binding to the antigen of the antibody and is also known as “antigen-binding fragment” or “antigen-binding portion”. In exemplary instances, the antigen binding antibody fragment is a Fab fragment or a F(ab′)2 fragment.

The term “ADCC” or “antibody-dependent cell-mediated cytotoxicity” or “antibody-dependent cellular cytotoxicity” refers to the mechanism by which an effector cell of the immune system (e.g., natural killer cells (NK cells), macrophages, neutrophils, eosinophils) actively lyses a target cell, whose membrane-surface antigens have been bound by specific antibodies. ADCC is a part of the adaptive immune response and occurs when antigen-specific antibodies bind to (1) the membrane-surface antigens on a target cell through its antigen-binding regions and (2) to Fc receptors on the surface of the effector cells through its Fc region. Binding of the Fc region of the antibody to the Fc receptor causes the effector cells to release cytotoxic factors that lead to death of the target cell (e.g., through cell lysis or cellular degranulation).

The term “ADCC activity” or “ADCC level” refers to the extent to which ADCC is activated or stimulated. Methods of measuring or determining the ADCC level of an antibody composition, including commercially available assays and kits for measuring or determining the ADCC level, are well-known in the art, as described, Yamashita et al., Scientific Reports 6: article number 19772 (2016), doi:10.1038/srep19772); Kantakamalakul et al., “A novel EGFP-CEM-NKr flow cytometric method for measuring antibody dependent cell mediated-cytotoxicity (ADCC) activity in HIV-1 infected individuals”, J Immunol Methods 315 (Issues 1-2): 1-10; (2006); Gomez-Roman et al., “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies”, Molec Immunology 38 (Issues 12-13): 1512-1517 (2011); and Mata et al., “Effects of cryopreservation on effector cells for antibody dependent cell-mediated cytotoxicity (ADCC) and natural killer (NK) cell activity in ⁵¹Cr-release and CD107a assays”, J Immunol Methods 406: 1-9 (2014); all herein incorporated by reference for all purposes. The term “ADCC Assay” or “FcyR reporter gene assay” refers to an assay, kit or method useful to determine the ADCC activity of an antibody. Exemplary methods of measuring or determining the ADCC activity of an antibody in the methods described herein include the ADCC assay described in the Example 2 or the ADCC Reporter Assay commercially available from Promega (Catalog No. G7010 and G7018). In some embodiments, ADCC activity is measured or determined using a calcein release assay containing one or more of the following: a FcγRIIa (158V)-expressing NK92(M1) cells as effector cells and HCC2218 cells or WIL2-S cells as target cells labeled with calcein-AM.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

### Methods of Increasing Sialic Acid Content

The methods described herein comprise expressing an α2,6-sialyltransferase 1 (ST6) or both ST6 and β1,4 galactosyltransferase 1 (B4GALT1) in the CHO cell to increase the sialic acid content of a therapeutic protein (e.g., an antibody).

In some embodiments, the method comprises introducing a polynucleotide sequence encoding ST6 into the CHO cell. In some embodiments, the method comprises introducing a polynucleotide sequences encoding both B4GALT1 and ST6 into the CHO cell. The polynucleotide and protein sequences for ST6 and B4GALT1 used for the present invention are set forth in Table 1 below.

TABLE 1

| | |
|---|---|
| SEQ ID | Human ST6 |
| NO: 1 | ATGATTCACACCAACCTGAAGAAAAAGTTCAGCTGCTGCGTCCTGGTCTTTC |
| | TTCTGTTTGCAGTCATCTGTGTGTGGAAGGAAAAGAAGAAAGGGAGTTACT |
| | ATGATTCCTTTAAATTGCAAACCAAGGAATTCCAGGTGTTAAAGAGTCTGG |
| | GGAAATTGGCCATGGGGTCTGATTCCCAGTCTGTATCCTCAAGCAGCACCCA |
| | GGACCCCCACAGGGGCCGCCAGACCCTCGGCAGTCTCAGAGGCCTAGCCAA |
| | GGCCAAACCAGAGGCCTCCTTCCAGGTGTGGAACAAGGACAGCTCTTCCAA |
| | AAACCTTATCCCTAGGCTGCAAAAGATCTGGAAGAATTACCTAAGCATGAA |
| | CAAGTACAAAGTGTCCTACAAGGGGCCAGGACCAGGCATCAAGTTCAGTGC |
| | AGAGGCCCTGCGCTGCCACCTCCGGGACCATGTGAATGTATCCATGGTAGA |
| | GGTCACAGATTTTCCCTTCAATACCTCTGAATGGGAGGGTTATCTGCCCAAG |
| | GAGAGCATTAGGACCAAGGCTGGGCCTTGGGGCAGGTGTGCTGTTGTGTCG |
| | TCAGCGGGATCTCTGAAGTCCTCCCAACTAGGCAGAGAAATCGATGATCAT |
| | GACGCAGTCCTGAGGTTTAATGGGGCACCCACAGCCAACTTCCAACAAGAT |
| | GTGGGCACAAAAACTACCATTCGCCTGATGAACTCTCAGTTGGTTACCACA |
| | GAGAAGCGCTTCCTCAAAGACAGTTTGTACAATGAAGGAATCCTAATTGTA |
| | TGGGACCCATCTGTATACCACTCAGATATCCCAAAGTGGTACCAGAATCCG |
| | GATTATAATTTCTTTAACAACTACAAGACTTATCGTAAGCTGCACCCCAATC |
| | AGCCCTTTTACATCCTCAAGCCCCAGATGCCTTGGGAGCTATGGGACATTCT |
| | TCAAGAAATCTCCCCAGAAGAGATTCAGCCAAACCCCCCATCCTCTGGGAT |
| | GCTTGGTATCATCATCATGATGACGCTGTGTGACCAGGTGGATATTTATGAG |
| | TTCCTCCCATCCAAGCGCAAGACTGACGTGTGCTACTACTACCAGAAGTTCT |
| | TCGATAGTGCCTGCACGATGGGTGCCTACCACCCGCTGCTCTATGAGAAGA |
| | ATTTGGTGAAGCATCTCAACCAGGGCACAGATGAGGACATCTACCTGCTTG |
| | GAAAAGCCACACTGCCTGGCTTCCGGACCATTCACTGCTAG |
| SEQ ID | Human ST6 |
| NO: 2 | MIHTNLKKKFSCCVLVFLLFAVICVWKEKKKGSYYDSFKLQTKEFQVLKSLGK |
| | LAMGSDSQSVSSSSTQDPHRGRQTLGSLRGLAKAKPEASFQVWNKDSSSKNLIP |
| | RLQKIWKNYLSMNKYKVSYKGPGPGIKFSAEALRCHLRDHVNVSMVEVTDFPF |
| | NTSEWEGYLPKESIRTKAGPWGRCAVVSSAGSLKSSQLGREIDDHDAVLRFNG |
| | APTANFQQDVGTKTTIRLMNSQLVTTEKRFLKDSLYNEGILIVWDPSVYHSDIP |
| | KWYQNPDYNFFNNYKTYRKLHPNQPFYILKPQMPWELWDILQEISPEEIQPNPP |
| | SSGMLGIIIMMTLCDQVDIYEFLPSKRKTDVCYYYQKFFDSACTMGAYHPLLYE |
| | KNLVKHLNQGTDEDIYLLGKATLPGFRTIHC |
| SEQ ID | Human ST6 variant 4 |
| NO: 3 | ATGATTCACACCAACCTGAAGAAAAAGTTCAGCTGCTGCGTCCTGGTCTTTC |
| | TTCTGTTTGCAGTCATCTGTGTGTGGAAGGAAAAGAAGAAAGGGAGTTACT |
| | ATGATTCCTTTAAATTGCAAACCAAGGAATTCCAGGTGTTAAAGAGTCTGG |
| | GGAAATTGGCCATGGGGTCTGATTCCCAGTCTGTATCCTCAAGCAGCACCCA |
| | GGACCCCCACAGGGGCCGCCAGACCCTCGGCAGTCTCAGAGGCCTAGCCAA |
| | GGCCAAACCAGAGGCCTCCTTCCAGGTGTGGAACAAGGACAGCTCTTCCAA |
| | AAACCTTATCCCTAGGCTGCAAAAGATCTGGAAGAATTACCTAAGCATGAA |
| | CAAGTACAAAGTGTCCTACAAGGGGCCAGGACCAGGCATCAAGTTCAGTGC |
| | AGAGGCCCTGCGCTGCCACCTCCGGGACCATGTGAATGTATCCATGGTAGA |
| | GGTCACAGATTTTCCCTTCAATACCTCTGAATGGGAGGGTTATCTGCCCAAG |
| | GAGAGCATTAGGACCAAGGCTGGGCCTTGGGGCAGGTGTGCTGTTGTGTCG |
| | TCAGCGGGATCTCTGAAGTCCTCCCAACTAGGCAGAGAAATCGATGATCAT |
| | GACGCAGTCCTGAGGTTTAATGGGGCACCCACAGCCAACTTCCAACAAGAT |
| | GTGGGCACAAAAACTACCATTCGCCTGATGAACTCTCAGTTGGTTACCACA |
| | GAGAAGCGCTTCCTCAAAGACAGTTTGTACAATGAAGGAATCCTAATTGTA |
| | TGGGACCCATCTGTATACCACTCAGATATCCCAAAGTGGTACCAGAATCCG |
| | GATTATAATTTCTTTAACAACTACAAGACTTATCGTAAGCTGCACCCCAATC |
| | AGCCCTTTTACATCCTCAAGCCCCAGATGCCTTGGGAGCTATGGGACATTCT |
| | TCAAGAAATCTCCCCAGAAGAGATTCAGCCAAACCCCCCATCCTCTGGGAT |
| | GCTTGGTATCATCATCATGATGACGCTGTGTGACCAGGTGGATATTTATGAG |
| | TTCCTCCCATCCAAGCGCAAGACTGACGTGTGCTACTACTACCAGAAGTTCT |
| | TCGATAGTGCCTGCACGATGGGTGCCTACCACCCGCTGCTCTATGAGAAGA |
| | ATTTGGTGAAGCATCTCAACCAGGGCACAGATGAGGACATCTACCTGCTTG |
| | GAAAAGCCACACTGCCTGGCTTCCGGACCATTCACTGCTAA |
| SEQ ID | Human ST6 variant 4 |
| NO: 4 | MIHTNLKKKFSCCVLVFLLFAVICVWKEKKKGSYYDSFKLQTKEFQVLKSLGK |
| | LAMGSDSQSVSSSSTQDPHRGRQTLGSLRGLAKAKPEASFQVWNKDSSSKNLIP |
| | RLQKIWKNYLSMNKYKVSYKGPGPGIKFSAEALRCHLRDHVNVSMVEVTDFPF |
| | NTSEWEGYLPKESIRTKAGPWGRCAVVSSAGSLKSSQLGREIDDHDAVLRFNG |
| | APTANFQQDVGTKTTIRLMNSQLVTTEKRFLKDSLYNEGILIVWDPSVYHSDIP |
| | KWYQNPDYNFFNNYKTYRKLHPNQPFYILKPQMPWELWDILQEISPEEIQPNPP |
| | SSGMLGIIIMMTLCDQVDIYEFLPSKRKTDVCYYYQKFFDSACTMGAYHPLLYE |
| | KNLVKHLNQGTDEDIYLLGKATLPGFRTIHC |

| | |
|---|---|
| SEQ ID | Mouse ST6 variant 1 |
| NO: 5 | ATGATTCATACCAACTTGAAGAGAAAGTTCAGCTGCTTTGTCCTGGTCTTTC |
| | TCCTGTTTGCCATCATCTGCGTGTGGAAGAAAGGGAGCGACTATGAGGCTCT |
| | TACATTGCAAGCCAAGGTATTCCAGATGCCGAAGAGCCAGGAGAAAGTGGC |
| | CGTGGGGCCTGCTCCCCAGGCTGTGTTCTCAAACAGCAAACAAGACCCTAA |
| | GGAAGGCGTTCAGATCCTCAGTTACCCCAGGGTCACAGCCAAGGTCAAGCC |
| | ACAGCCCTCCTTGCAGGTGTGGGACAAGGACTCCACATACTCAAAACTTAA |
| | CCCCAGGCTGCTGAAGATCTGGAGGAACTATCTGAACATGAATAAATATAA |
| | AGTGTCCTACAAGGGGCCGGGACCAGGAGTCAAGTTCAGCGTAGAGGCGCT |
| | GCGCTGCCACCTTCGAGACCACGTGAATGTGTCTATGATAGAGGCCACAGA |
| | TTTTCCCTTCAACACCACTGAATGGGAGGGTTACCTGCCCAAGGAGAACTTC |
| | AGAACCAAGGCTGGGCCTTGGCATAAGTGTGCCGTCGTGTCTTCTGCAGGA |
| | TCTCTGAAGAACTCCCAGCTGGGTCGAGAGATTGATAATCATGATGCGGTC |
| | CTGAGGTTTAATGGGGCACCTACAGACAACTTCCAACAGGATGTGGGCACA |
| | AAAACTACCATCCGCCTAGTGAACTCTCAGTTAGTCACCACAGAAAAGCGC |
| | TTCCTGAAGGACAGTTTGTACACCGAAGGAATCCTGATTCTGTGGGACCCAT |
| | CTGTGTATCATGCAGACATTCCGCAGTGGTATCAGAAGCCAGACTACAACTT |
| | CTTCGAAACCTATAAGAGTTACCGAAGGCTTCACCCCAGCCAGCCTTTTTAC |
| | ATCCTCAAGCCCCAGATGCCATGGGAACTATGGGACATCATTCAGGAAATC |
| | TCTCCAGATCTGATTCAGCCGAATCCCCCATCCTCCGGCATGCTGGGTATCA |
| | TCATTATGATGACGCTGTGTGACCAAGTTGATATTTACGAGTTCCTCCCATC |
| | CAAGCGCAAGACAGATGTGTGCTACTATCACCAGAAGTTCTTTGACAGCGC |
| | CTGCACGATGGGTGCCTACCATCCGCTCCTCTTCGAGAAGAATATGGTGAA |
| | GCATCTCAATGAGGGAACAGATGAAGACATTTATTTGTTTGGGAAAGCTAC |
| | CCTGTCTGGCTTCCGGAACAATCGCTGTTGA |
| SEQ ID | Mouse ST6 variant 1 |
| NO: 6 | MIHTNLKRKFSCFVLVFLLFAIICVWKKGSDYEALTLQAKVFQMPKSQEKVAV |
| | GPAPQAVFSNSKQDPKEGVQILSYPRVTAKVKPQPSLQVWDKDSTYSKLNPRL |
| | LKIWRNYLNMNKYKVSYKGPGPGVKFSVEALRCHLRDHVNVSMIEATDFPFN |
| | TTEWEGYLPKENFRTKAGPWHKCAVVSSAGSLKNSQLGREIDNHDAVLRFNG |
| | APTDNFQQDVGTKTTIRLVNSQLVTTEKRFLKDSLYTEGILILWDPSVYHADIPQ |
| | WYQKPDYNFFETYKSYRRLHPSQPFYILKPQMPWELWDIIQEISPDLIQPNPPSS |
| | GMLGIIIMMTLCDQVDIYEFLPSKRKTDVCYYHQKFFDSACTMGAYHPLLFEK |
| | NMVKHLNEGTDEDIYLFGKATLSGFRNNRC |
| SEQ ID | Mouse ST6 variant 2 |
| NO: 7 | ATGATTCATACCAACTTGAAGAGAAAGTTCAGCTGCTTTGTCCTGGTCTTTC |
| | TCCTGTTTGCCATCATCTGCGTGTGGAAGAAAGGGAGCGACTATGAGGCTCT |
| | TACATTGCAAGCCAAGGTATTCCAGATGCCGAAGAGCCAGGAGAAAGTGGC |
| | CGTGGGGCCTGCTCCCCAGGCTGTGTTCTCAAACAGCAAACAAGACCCTAA |
| | GGAAGGCGTTCAGATCCTCAGTTACCCCAGGGTCACAGCCAAGGTCAAGCC |
| | ACAGCCCTCCTTGCAGGTGTGGGACAAGGACTCCACATACTCAAAACTTAA |
| | CCCCAGGCTGCTGAAGATCTGGAGGAACTATCTGAACATGAATAAATATAA |
| | AGTGTCCTACAAGGGGCCGGGACCAGGAGTCAAGTTCAGCGTAGAGGCGCT |
| | GCGCTGCCACCTTCGAGACCACGTGAATGTGTCTATGATAGAGGCCACAGA |
| | TTTTCCCTTCAACACCACTGAATGGGAGGGTTACCTGCCCAAGGAGAACTTC |
| | AGAACCAAGGCTGGGCCTTGGCATAAGTGTGCCGTCGTGTCTTCTGCAGGA |
| | TCTCTGAAGAACTCCCAGCTGGGTCGAGAGATTGATAATCATGATGCGGTC |
| | CTGAGGTTTAATGGGGCACCTACAGACAACTTCCAACAGGATGTGGGCACA |
| | AAAACTACCATCCGCCTAGTGAACTCTCAGTTAGTCACCACAGAAAAGCGC |
| | TTCCTGAAGGACAGTTTGTACACCGAAGGAATCCTGATTCTGTGGGACCCAT |
| | CTGTGTATCATGCAGACATTCCGCAGTGGTATCAGAAGCCAGACTACAACTT |
| | CTTCGAAACCTATAAGAGTTACCGAAGGCTTCACCCCAGCCAGCCTTTTTAC |
| | ATCCTCAAGCCCCAGATGCCATGGGAACTATGGGACATCATTCAGGAAATC |
| | TCTCCAGATCTGATTCAGCCGAATCCCCCATCCTCCGGCATGCTGGGTATCA |
| | TCATTATGATGACGCTGTGTGACCAAGTTGATATTTACGAGTTCCTCCCATC |
| | CAAGCGCAAGACAGATGTGTGCTACTATCACCAGAAGTTCTTTGACAGCGC |
| | CTGCACGATGGGTGCCTACCATCCGCTCCTCTTCGAGAAGAATATGGTGAA |
| | GCATCTCAATGAGGGAACAGATGAAGACATTTATTTGTTTGGGAAAGCTAC |
| | CCTGTCTGGCTTCCGGAACAATCGCTGTTGA |
| SEQ ID | Mouse ST6 variant 2 |
| NO: 8 | MIHTNLKRKFSCFVLVFLLFAIICVWKKGSDYEALTLQAKVFQMPKSQEKVAV |
| | GPAPQAVFSNSKQDPKEGVQILSYPRVTAKVKPQPSLQVWDKDSTYSKLNPRL |
| | LKIWRNYLNMNKYKVSYKGPGPGVKFSVEALRCHLRDHVNVSMIEATDFPFN |
| | TTEWEGYLPKENFRTKAGPWHKCAVVSSAGSLKNSQLGREIDNHDAVLRFNG |
| | APTDNFQQDVGTKTTIRLVNSQLVTTEKRFLKDSLYTEGILILWDPSVYHADIPQ |
| | WYQKPDYNFFETYKSYRRLHPSQPFYILKPQMPWELWDIIQEISPDLIQPNPPSS |
| | GMLGIIIMMTLCDQVDIYEFLPSKRKTDVCYYHQKFFDSACTMGAYHPLLFEK |
| | NMVKHLNEGTDEDIYLFGKATLSGFRNNRC |
| SEQ ID | Mouse ST6 variant 3 |
| NO: 9 | ATGATTCATACCAACTTGAAGAGAAAGTTCAGCTGCTTTGTCCTGGTCTTTC |
| | TCCTGTTTGCCATCATCTGCGTGTGGAAGAAAGGGAGCGACTATGAGGCTCT |
| | TACATTGCAAGCCAAGGTATTCCAGATGCCGAAGAGCCAGGAGAAAGTGGC |
| | CGTGGGGCCTGCTCCCCAGGCTGTGTTCTCAAACAGCAAACAAGACCCTAA |
| | GGAAGGCGTTCAGATCCTCAGTTACCCCAGGGTCACAGCCAAGGTCAAGCC |
| | ACAGCCCTCCTTGCAGGTGTGGGACAAGGACTCCACATACTCAAAACTTAA |
| | CCCCAGGCTGCTGAAGATCTGGAGGAACTATCTGAACATGAATAAATATAA |
| | AGTGTCCTACAAGGGGCCGGGACCAGGAGTCAAGTTCAGCGTAGAGGCGCT |
| | GCGCTGCCACCTTCGAGACCACGTGAATGTGTCTATGATAGAGGCCACAGA |
| | TTTTCCCTTCAACACCACTGAATGGGAGGGTTACCTGCCCAAGGAGAACTTC |
| | AGAACCAAGGCTGGGCCTTGGCATAAGTGTGCCGTCGTGTCTTCTGCAGGA |
| | TCTCTGAAGAACTCCCAGCTGGGTCGAGAGATTGATAATCATGATGCGGTC |
| | CTGAGGTTTAATGGGGCACCTACAGACAACTTCCAACAGGATGTGGGCACA |
| | AAAACTACCATCCGCCTAGTGAACTCTCAGTTAGTCACCACAGAAAAGCGC |
| | TTCCTGAAGGACAGTTTGTACACCGAAGGAATCCTGATTCTGTGGGACCCAT |
| | CTGTGTATCATGCAGACATTCCGCAGTGGTATCAGAAGCCAGACTACAACTT |
| | CTTCGAAACCTATAAGAGTTACCGAAGGCTTCACCCCAGCCAGCCTTTTTAC |
| | ATCCTCAAGCCCCAGATGCCATGGGAACTATGGGACATCATTCAGGAAATC |
| | TCTCCAGATCTGATTCAGCCGAATCCCCCATCCTCCGGCATGCTGGGTATCA |
| | TCATTATGATGACGCTGTGTGACCAAGTTGATATTTACGAGTTCCTCCCATC |
| | CAAGCGCAAGACAGATGTGTGCTACTATCACCAGAAGTTCTTTGACAGCGC |
| | CTGCACGATGGGTGCCTACCATCCGCTCCTCTTCGAGAAGAATATGGTGAA |
| | GCATCTCAATGAGGGAACAGATGAAGACATTTATTTGTTTGGGAAAGCTAC |
| | CCTGTCTGGCTTCCGGAACAATCGCTGTTGA |
| SEQ ID | Mouse ST6 variant 3 |
| NO: 10 | MIHTNLKRKFSCFVLVFLLFAIICVWKKGSDYEALTLQAKVFQMPKSQEKVAV |
| | GPAPQAVFSNSKQDPKEGVQILSYPRVTAKVKPQPSLQVWDKDSTYSKLNPRL |
| | LKIWRNYLNMNKYKVSYKGPGPGVKFSVEALRCHLRDHVNVSMIEATDFPFN |
| | TTEWEGYLPKENFRTKAGPWHKCAVVSSAGSLKNSQLGREIDNHDAVLRFNG |
| | APTDNFQQDVGTKTTIRLVNSQLVTTEKRFLKDSLYTEGILILWDPSVYHADIPQ |
| | WYQKPDYNFFETYKSYRRLHPSQPFYILKPQMPWELWDIIQEISPDLIQPNPPSS |
| | GMLGIIIMMTLCDQVDIYEFLPSKRKTDVCYYHQKFFDSACTMGAYHPLLFEK |
| | NMVKHLNEGTDEDIYLFGKATLSGFRNNRC |
| SEQ ID | Rat ST6 variant 3 |
| NO: 11 | ATGATTCATACCAACTTGAAGAAAAAGTTCAGCCTCTTCATCCTGGTCTTTC |
| | TCCTGTTCGCAGTCATCTGTGTTTGGAAGAAAGGGAGCGACTATGAGGCCCT |
| | TACACTGCAAGCCAAGGAATTCCAGATGCCCAAGAGCCAGGAGAAAGTGG |
| | CCATGGGGTCTGCTTCCCAGGTTGTGTTCTCAAACAGCAAGCAAGACCCTAA |
| | GGAAGACATTCCAATCCTCAGTTACCACAGGGTCACAGCCAAGGTCAAACC |
| | ACAGCCTTCCTTCCAGGTGTGGGACAAGGACTCCACATACTCAAAACTTAA |
| | CCCCAGGCTGCTGAAGATCTGGAGAAACTATCTGAACATGAACAAATATAA |
| | AGTATCCTACAAGGGACCGGGGCCAGGAGTCAAGTTCAGCGTAGAAGCACT |
| | GCGTTGCCACCTTCGAGACCATGTGAACGTGTCTATGATAGAGGCCACAGA |
| | TTTTCCCTTCAACACCACTGAGTGGGAGGGTTACCTGCCCAAGGAGAACTTT |
| | AGAACCAAGGTTGGGCCTTGGCAAAGGTGTGCCGTCGTCTCTTCTGCAGGA |
| | TCTCTGAAAAACTCCCAGCTTGGTCGAGAGATTGATAATCATGATGCAGTTC |
| | TGAGGTTTAATGGGGCCCCTACCGACAACTTCCAACAGGATGTGGGCTCAA |
| | AAACTACCATTCGCCTAATGAACTCTCAGTTAGTCACCACAGAAAAGCGCTT |
| | CCTCAAGGACAGTTTGTACACCGAAGGAATCCTAATTGTATGGGACCCATC |
| | CGTGTATCATGCAGATATCCCAAAGTGGTATCAGAAACCAGACTACAATTT |
| | CTTCGAAACCTATAAGAGTTACCGAAGGCTGAACCCCAGCCAGCCATTTTAT |
| | ATCCTCAAGCCCCAGATGCCATGGGAACTGTGGGACATCATTCAGGAAATC |
| | TCTGCAGATCTGATTCAGCCAAATCCCCCATCCTCCGGCATGCTGGGTATCA |
| | TCATCATGATGACGCTGTGTGACCAGGTAGATATTTACGAGTTCCTCCCATC |
| | CAAGCGCAAGACGGACGTGTGCTATTATCACCAAAAGTTCTTTGACAGCGC |
| | TTGCACGATGGGTGCCTACCACCCGCTCCTCTTCGAGAAGAATATGGTGAA |
| | GCATCTCAATGAGGGAACAGATGAAGACATTTATTTGTTTGGGAAAGCCAC |
| | CCTTTCTGGCTTCCGGAACATTCGTTGTTGA |
| SEQ ID | Rat ST6 variant 3 |
| NO: 12 | MIHTNLKKKFSLFILVFLLFAVICVWKKGSDYEALTLQAKEFQMPKSQEKVAM |
| | GSASQVVFSNSKQDPKEDIPILSYHRVTAKVKPQPSFQVWDKDSTYSKLNPRLL |
| | KIWRNYLNMNKYKVSYKGPGPGVKFSVEALRCHLRDHVNVSMIEATDFPFNT |
| | TEWEGYLPKENFRTKVGPWQRCAVVSSAGSLKNSQLGREIDNHDAVLRFNGA |
| | PTDNFQQDVGSKTTIRLMNSQLVTTEKRFLKDSLYTEGILIVWDPSVYHADIPK |
| | WYQKPDYNFFETYKSYRRLNPSQPFYILKPQMPWELWDIIQEISADLIQPNPPSS |
| | GMLGIIIMMTLCDQVDIYEFLPSKRKTDVCYYHQKFFDSACTMGAYHPLLFEK |
| | NMVKHLNEGTDEDIYLFGKATLSGFRNIRC |
| SEQ ID | Human B4GALT1 |
| NO: 13 | ATGAGGCTTCGGGAGCCGCTCCTGAGCGGCAGCGCCGCGATGCCAGGCGCG |
| | TCCCTACAGCGGGCCTGCCGCCTGCTCGTGGCCGTCTGCGCTCTGCACCTTG |
| | GCGTCACCCTCGTTTACTACCTGGCTGGCCGCGACCTGAGCCGCCTGCCCCA |
| | ACTGGTCGGAGTCTCCACACCGCTGCAGGGCGGCTCGAACAGTGCAGCAGC |
| | TATAGGCCAGTCAAGCGGTGAACTGAGGACTGGGGGTGCCCGACCACCACC |
| | TCCACTCGGCGCTTCATCCCAACCACGTCCTGGGGGAGACTCCAGCCCAGTC |
| | GTGGATTCTGGCCCTGGCCCCGCTAGCAACTTGACCTCGGTCCCAGTGCCCC |
| | ACACCACCGCACTGTCGCTGCCCGCCTGCCCTGAGGAGTCCCCGCTGCTTGT |
| | GGGCCCCATGCTGATTGAGTTTAACATGCCTGTGGACCTGGAGCTCGTGGCA |
| | AAGCAGAACCCAAATGTGAAGATGGGCGGCCGCTATGCCCCCAGGGACTGC |
| | GTCTCTCCTCACAAGGTGGCCATCATCATTCCATTCCGCAACCGGCAGGAGC |
| | ACCTCAAGTACTGGCTATATTATTTGCACCCAGTCCTGCAGCGCCAGCAGCT |
| | GGACTATGGCATCTATGTTATCAACCAGGCGGGAGACACTATATTCAATCGT |
| | GCTAAGCTCCTCAATGTTGGCTTTCAAGAAGCCTTGAAGGACTATGACTACA |
| | CCTGCTTTGTGTTTAGTGACGTGGACCTCATTCCAATGAATGACCATAATGC |
| | GTACAGGTGTTTTTCACAGCCACGGCACATTTCCGTTGCAATGGATAAGTTT |
| | GGATTCAGCCTACCTTATGTTCAGTATTTTGGAGGTGTCTCTGCTCTAAGTA |
| | AACAACAGTTTCTAACCATCAATGGATTTCCTAATAATTATTGGGGCTGGGG |
| | AGGAGAAGATGATGACATTTTTAACAGATTAGTTTTTAGAGGCATGTCTATA |
| | TCTCGCCCAAATGCTGTGGTCGGGAGGTGTCGCATGATCCGCCACTCAAGA |
| | GACAAGAAAAATGAACCCAATCCTCAGAGGTTTGACCGAATTGCACACACA |
| | AAGGAGACAATGCTCTCTGATGGTTTGAACTCACTCACCTACCAGGTGCTGG |
| | ATGTACAGAGATACCCATTGTATACCCAAATCACAGTGGACATCGGGACAC |
| | CGAGCTAG |
| SEQ ID | Human B4GALT1 |
| NO: 14 | MRLREPLLSGSAAMPGASLQRACRLLVAVCALHLGVTLVYYLAGRDLSRLPQL |
| | VGVSTPLQGGSNSAAAIGQSSGELRTGGARPPPPLGASSQPRPGGDSSPVVDSGP |
| | GPASNLTSVPVPHTTALSLPACPEESPLLVGPMLIEFNMPVDLELVAKQNPNVK |
| | MGGRYAPRDCVSPHKVAIIIPFRNRQEHLKYWLYYLHPVLQRQQLDYGIYVIN |
| | QAGDTIFNRAKLLNVGFQEALKDYDYTCFVFSDVDLIPMNDHNAYRCFSQPRH |
| | ISVAMDKFGFSLPYVQYFGGVSALSKQQFLTINGFPNNYWGWGGEDDDIFNRL |
| | VFRGMSISRPNAVVGRCRMIRHSRDKKNEPNPQRFDRIAHTKETMLSDGLNSLT |
| | YQVLDVQRYPLYTQITVDIGTPS |
| SEQ ID | Human B4GALT1 Variant 2 |
| NO: 15 | ATGCCAGGCGCGTCCCTACAGCGGGCCTGCCGCCTGCTCGTGGCCGTCTGCG |
| | CTCTGCACCTTGGCGTCACCCTCGTTTACTACCTGGCTGGCCGCGACCTGAG |
| | CCGCCTGCCCCAACTGGTCGGAGTCTCCACACCGCTGCAGGGCGGCTCGAA |
| | CAGTGCCGCCGCCATCGGGCAGTCCTCCGGGGAGCTCCGGACCGGAGGGGC |
| | CCGGCCGCCGCCTCCTCTAGGCGCCTCCTCCCAGCCGCGCCCGGGTGGCGAC |
| | TCCAGCCCAGTCGTGGATTCTGGCCCTGGCCCCGCTAGCAACTTGACCTCGG |
| | TCCCAGTGCCCCACACCACCGCACTGTCGCTGCCCGCCTGCCCTGAGGAGTC |
| | CCCGCTGCTTGTGGGCCCCATGCTGATTGAGTTTAACATGCCTGTGGACCTG |
| | GAGCTCGTGGCAAAGCAGAACCCAAATGTGAAGATGGGCGGCCGCTATGCC |
| | CCCAGGGACTGCGTCTCTCCTCACAAGGTGGCCATCATCATTCCATTCCGCA |
| | ACCGGCAGGAGCACCTCAAGTACTGGCTATATTATTTGCACCCAGTCCTGCA |
| | GCGCCAGCAGCTGGACTATGGCATCTATGTTATCAACCAGGCGGGAGACAC |
| | TATATTCAATCGTGCTAAGCTCCTCAATGTTGGCTTTCAAGAAGCCTTGAAG |
| | GACTATGACTACACCTGCTTTGTGTTTAGTGACGTGGACCTCATTCCAATGA |
| | ATGACCATAATGCGTACAGGTGTTTTTCACAGCCACGGCACATTTCCGTTGC |
| | AATGGATAAGTTTGGATTCAGCCTACCTTATGTTCAGTATTTTGGAGGTGTC |
| | TCTGCTCTAAGTAAACAACAGTTTCTAACCATCAATGGATTTCCTAATAATT |
| | ATTGGGGCTGGGGAGGAGAAGATGATGACATTTTTAACAGATTAGTTTTTA |
| | GAGGCATGTCTATATCTCGCCCAAATGCTGTGGTCGGGAGGTGTCGCATGAT |
| | CCGCCACTCAAGAGACAAGAAAAATGAACCCAATCCTCAGAGGTTTGACCG |
| | AATTGCACACACAAAGGAGACAATGCTCTCTGATGGTTTGAACTCACTCAC |
| | CTACCAGGTGCTGGATGTACAGAGATACCCATTGTATACCCAAATCACAGT |
| | GGACATCGGGACACCGAGCTAG |
| SEQ ID | Human B4GALT1 Variant 2 |
| NO: 16 | MPGASLQRACRLLVAVCALHLGVTLVYYLAGRDLSRLPQLVGVS |
| | TPLQGGSNSAAAIGQSSGELRTGGARPPPPLGASSQPRPGGDSSPVVDSGPGPAS |
| | NLTSVPVPHTTALSLPACPEESPLLVGPMLIEFNMPVDLELVAKQNPNVKMGGR |
| | YAPRDCVSPHKVAIIIPFRNRQEHLKYWLYYLHPVLQRQQLDYGIYVINQAGDT |
| | IFNRAKLLNVGFQEALKDYDYTCFVFSDVDLIPMNDHNAYRCFSQPRHISVAM |
| | DKFGFSLPYVQYFGGVSALSKQQFLTINGFPNNYWGWGGEDDDIFNRLVFRGM |
| | SISRPNAVVGRCRMIRHSRDKKNEPNPQRFDRIAHTKETMLSDGLNSLTYQVLD |
| | VQRYPLYTQITVDIGTPS |
| SEQ ID | Human B4GALT1 Variant 3 |
| NO: 17 | ATGAGGCTTCGGGAGCCGCTCCTGAGCGGCAGCGCCGCGATGCCAGGCGCG |
| | TCCCTACAGCGGGCCTGCCGCCTGCTCGTGGCCGTCTGCGCTCTGCACCTTG |
| | GCGTCACCCTCGTTTACTACCTGGCTGGCCGCGACCTGAGCCGCCTGCCCCA |
| | ACTGGTCGGAGTCTCCACACCGCTGCAGGGCGGCTCGAACAGTGCCGCCGC |
| | CATCGGGCAGTCCTCCGGGGAGCTCCGGACCGGAGGGGCCCGGCCGCCGCC |
| | TCCTCTAGGCGCCTCCTCCCAGCCGCGCCCGGGTGGCGACTCCAGCCCAGTC |
| | GTGGATTCTGGCCCTGGCCCCGCTAGCAACTTGACCTCGGTCCCAGTGCCCC |
| | ACACCACCGCACTGTCGCTGCCCGCCTGCCCTGAGGAGTCCCCGCTGCTTGT |
| | GGGCCCCATGCTGATTGAGTTTAACATGCCTGTGGACCTGGAGCTCGTGGCA |
| | AAGCAGAACCCAAATGTGAAGATGGGCGGCCGCTATGCCCCCAGGGACTGC |
| | GTCTCTCCTCACAAGGTGGCCATCATCATTCCATTCCGCAACCGGCAGGAGC |
| | ACCTCAAGTACTGGCTATATTATTTGCACCCAGTCCTGCAGCGCCAGCAGCT |
| | GGACTATGGCATCTATGTTATCAACCAGGCGGGAGACACTATATTCAATCGT |
| | GCTAAGCTCCTCAATGTTGGCTTTCAAGAAGCCTTGAAGGACTATGACTACA |
| | CCTGCTTTGTGTTTAGTGACGTGGACCTCATTCCAATGAATGACCATAATGC |
| | GTACAGGTGTTTTTCACAGCCACGGCACATTTCCGTTGCAATGGATAAGTTT |
| | GGATTCAGATTAGTTTTTAGAGGCATGTCTATATCTCGCCCAAATGCTGTGG |
| | TCGGGAGGTGTCGCATGATCCGCCACTCAAGAGACAAGAAAAATGAACCCA |
| | ATCCTCAGAGGTTTGACCGAATTGCACACACAAAGGAGACAATGCTCTCTG |
| | ATGGTTTGAACTCACTCACCTACCAGGTGCTGGATGTACAGAGATACCCATT |
| | GTATACCCAAATCACAGTGGACATCGGGACACCGAGCTAG |
| SEQ ID | Human B4GALT1 Variant 3 |
| NO: 18 | MRLREPLLSGSAAMPGASLQRACRLLVAVCALHLGVTLVYYLAGRDLSRLPQL |
| | VGVSTPLQGGSNSAAAIGQSSGELRTGGARPPPPLGASSQPRPGGDSSPVVDSGP |
| | GPASNLTSVPVPHTTALSLPACPEESPLLVGPMLIEFNMPVDLELVAKQNPNVM |
| | GGRYAPRDCVSPHKVAIIIPFRNRQEHLKYWLYYLHPVLQRQQLDYGIYVINQA |
| | GDTIFNRAKLLNVGFQEALKDYDYTCFVFSDVDLIPMNDHNAYRCFSQPRHISV |
| | AMDKFGFRLVFRGMSISRPNAVVGRCRMIRHSRDKKNEPNPQRFDRIAHTKET |
| | MLSDGLNSLTYQVLDVQRYPLYTQITVDIGTPS |
| SEQ ID | Human B4GALT1 Variant 4 |
| NO: 19 | ATGAGGCTTCGGGAGCCGCTCCTGAGCGGCAGCGCCGCGATGCCAGGCGCG |
| | TCCCTACAGCGGGCCTGCCGCCTGCTCGTGGCCGTCTGCGCTCTGCACCTTG |
| | GCGTCACCCTCGTTTACTACCTGGCTGGCCGCGACCTGAGCCGCCTGCCCCA |
| | ACTGGTCGGAGTCTCCACACCGCTGCAGGGCGGCTCGAACAGTGCCGCCGC |
| | CATCGGGCAGTCCTCCGGGGAGCTCCGGACCGGAGGGGCCCGGCCGCCGCC |
| | TCCTCTAGGCGCCTCCTCCCAGCCGCGCCCGGGTGGCGACTCCAGCCCAGTC |
| | GTGGATTCTGGCCCTGGCCCCGCTAGCAACTTGACCTCGGTCCCAGTGCCCC |
| | ACACCACCGCACTGTCGCTGCCCGCCTGCCCTGAGGAGTCCCCGCTGCTTGT |
| | GGGCCCCATGCTGATTGAGTTTAACATGCCTGTGGACCTGGAGCTCGTGGCA |
| | AAGCAGAACCCAAATGTGAAGATGGGCGGCCGCTATGCCCCCAGGGACTGC |
| | GTCTCTCCTCACAAGGTGGCCATCATCATTCCATTCCGCAACCGGCAGGAGC |
| | ACCTCAAGTACTGGCTATATTATTTGCACCCAGTCCTGCAGCGCCAGCAGCT |
| | GGACTATGGCATCTATGTTATCAACCAGTATGAAAAAATAAGAAGACTCCT |
| | GTGGTGA |
| SEQ ID | Human B4GALT1 Variant 4 |
| NO: 20 | MRLREPLLSGSAAMPGASLQRACRLLVAVCALHLGVTLVYYLAGRDLSRLPQL |
| | VGVSTPLQGGSNSAAAIGQSSGELRTGGARPPPPLGASSQPRPGGDSSPVVDSGP |
| | GPASNLTSVPVPHTTALSLPACPEESPLLVGPMLIEFNMPVDLELVAKQNPNVK |
| | MGGRYAPRDCVSPHKVAIIIPFRNRQEHLKYWLYYLHPVLQRQQLDYGIYVIN |
| | QYEKIRRLLW |
| SEQ ID | CHO B4GALT1 |
| NO: 21 | ATGAGGTTTCTGAGGCCGGTCCTGGGCGGCAGCGCCGCGATGCCGGGCGCG |
| | ACCCTGCAGCGGGCCTGCCGCCTGCTCGTGGCCGTCTGCGCGCTGCACCTCG |
| | GCGTCACCCTGGTCTATTACCTCTCGGGCCGCGACCTGAGCCGCCTGCCTCA |
| | GCTAGTCGGGGTTTCCTCTACACTTCGGAGCGGCACGATCGGCGCCACCGCC |
| | AACAAGCAGCCCCCCGGCGCGCGGCCGCCGCCTCCTGTGGGAGTCTCCTCG |
| | AAGCCTCGCCCGGGTCCCGACTCCAGCCCTGGTACGGCTTTTGACCCCGGCC |
| | TGAAGAGCAACTGGACTTCGGTCCTAGTGCCCCCTACCACTGCACTGTTGAC |
| | GCTGCCCGCTTGCCCCGAGGAGTCCCCGCTGCTCGTTGGCCCCATGGTGATT |
| | GACTTTAATATTGCTGTGGACCTGGAGCTTTTGGCAAAGAAGAACCCAGAG |
| | ATAAAGATGGGCGGTCGTTATTCCCCCAAGGACTGTATTTCTCCTCACAAGG |
| | TGGCCATCATTATCCCATTTCGCAACCGGCAGGAGCACCTCAAATATTGGCT |
| | GTATTATTTGCATCCGGTCCTACAGCGCCAGCAACTGGACTATGGTATCTAC |
| | GTCATCAACCAGGCTGGAGACACCATGTTCAATCGAGCTAAGCTTCTCAAC |
| | ATAGGCTTCCAAGAGGCCTTGAAGGACCATGACTACAACTGCTTTGTGTTCA |
| | GTGATGTGGACCTTATTCCAATGGACGACCATAATGCCTACAGGTGTTTTTC |
| | ACAGCCCCGGCACATTTCTGTTGCAATGGACAAGTTTGGGTTTAGCCTGCCT |
| | TATGTTCAGTACTTTGGAGGCGTTTCTGCCCTCAGTAAACAACAGTTTCTTG |
| | CCATCAATGGATTTCCTAATAATTATTGGGGCTGGGGAGGAGAAGATGATG |
| | ACATTTTTAACAGAATAGTTCATAAGGGAATGTCTATATCACGCCCAAATGC |
| | TGTAGTTGGAAGATGTCGCATGATACGACACTCAAGAGACAAGAAAAATGA |
| | GCCCAATCCTCAGAGGTTTGACCGGATTGCTCACACAAAGGAAACTATGCG |
| | CTTTGATGGTTTGAACTCACTTACCTACCAGGTGTTGAACGTAGAGAGATAC |
| | CCATTATATACCAAAATCACAGTGGACATCGGGACACCAAGATAGCATTGT |
| | GGAACAAATAACAGACCTGAAAATGGCCAGGGGCTCAGA |
| SEQ ID | CHO B4GALT1 |
| NO: 22 | MRFLRPVLGGSAAMPGATLQRACRLLVAVCALHLGVTLVYYLSGRDLSRLPQ |
| | LVGVSSTLRSGTIGATANKQPPGARPPPPVGVSSKPRPGPDSSPGTAFDPGLKSN |
| | WTSVLVPPTTALLTLPACPEESPLLVGPMVIDFNIAVDLELLAKKNPEIKMGGR |
| | YSPKDCISPHKVAIIIPFRNRQEHLKYWLYYLHPVLQRQQLDYGIYVINQAGDT |
| | MFNRAKLLNIGFQEALKDHDYNCFVFSDVDLIPMDDHNAYRCFSQPRHISVAM |
| | DKFGFSLPYVQYFGGVSALSKQQFLAINGFPNNYWGWGGEDDDIFNRIVHKGM |
| | SISRPNAVVGRCRMIRHSRDKKNEPNPQRFDRIAHTKETMRFDGLNSLTYQVLN |
| | VERYPLYTKITVDIGTPR |
| SEQ ID | Mouse B4GALT1 Variant 1 |
| NO: 23 | ATGAGGTTTCGTGAGCAGTTCCTGGGCGGCAGCGCCGCGATGCCGGGCGCG |
| | ACCCTGCAGCGGGCCTGCCGCCTGCTCGTGGCCGTCTGCGCGCTGCACCTCG |
| | GCGTCACCCTCGTCTATTACCTCTCTGGCCGCGATCTGAGCCGCCTGCCCCA |
| | GTTGGTCGGAGTCTCCTCTACACTGCAGGGCGGCACGAACGGCGCCGCAGC |
| | CAGCAAGCAGCCCCCAGGAGAGCAGCGGCCGCGGGGTGCGCGGCCGCCGC |
| | CTCCTTTAGGCGTCTCCCCGAAGCCTCGCCCGGGTCTCGACTCCAGCCCTGG |
| | TGCAGCTTCTGGCCCCGGCTTGAAGAGCAACTTGTCTTCGTTGCCAGTGCCC |
| | ACCACCACTGGACTGTTGTCGCTGCCAGCTTGCCCTGAGGAGTCCCCGCTGC |
| | TCGTTGGCCCCATGCTGATTGACTTTAATATTGCTGTGGATCTGGAGCTTTTG |
| | GCAAAGAAGAACCCAGAGATAAAGACGGGCGGCCGTTACTCCCCCAAGGA |
| | CTGTGTCTCTCCTCACAAGGTGGCCATCATCATCCCATTCCGTAACCGGCAG |
| | GAGCATCTCAAATACTGGCTGTATTATTTGCATCCCATCCTTCAGCGCCAGC |
| | AACTCGACTATGGCATCTACGTCATCAATCAGGCTGGAGACACCATGTTCA |
| | ATCGAGCTAAGCTGCTCAATATTGGCTTTCAAGAGGCCTTGAAGGACTATG |
| | ATTACAACTGCTTTGTGTTCAGTGATGTGGACCTCATTCCGATGGACGACCG |
| | TAATGCCTACAGGTGTTTTTCGCAGCCACGGCACATTTCTGTTGCAATGGAC |
| | AAGTTCGGGTTTAGCCTGCCATATGTTCAGTATTTTGGAGGTGTCTCTGCTCT |
| | CAGTAAACAACAGTTTCTTGCCATCAATGGATTCCCTAATAATTATTGGGGT |
| | TGGGGAGGAGAAGATGACGACATTTTTAACAGATTAGTTCATAAAGGCATG |
| | TCTATATCACGTCCAAATGCTGTAGTAGGGAGGTGTCGAATGATCCGGCATT |
| | CAAGAGACAAGAAAAATGAGCCCAATCCTCAGAGGTTTGACCGGATCGCAC |
| | ATACAAAGGAAACGATGCGCTTCGATGGTTTGAACTCACTTACCTACAAGG |
| | TGTTGGATGTACAGAGATACCCGTTATATACCCAAATCACAGTGGACATCG |
| | GGACACCGAGATAG |
| SEQ ID | Mouse B4GALT1 Variant 1 |
| NO: 24 | MRFREQFLGGSAAMPGATLQRACRLLVAVCALHLGVTLVYYLSGRDLSRLPQ |
| | LVGVSSTLQGGTNGAAASKQPPGEQRPRGARPPPPLGVSPKPRPGLDSSPGAAS |
| | GPGLKSNLSSLPVPTTTGLLSLPACPEESPLLVGPMLIDFNIAVDLELLAKKNPEI |
| | KTGGRYSPKDCVSPHKVAIIIPFRNRQEHLKYWLYYLHPILQRQQLDYGIYVINQ |
| | AGDTMFNRAKLLNIGFQEALKDYDYNCFVFSDVDLIPMDDRNAYRCFSQPRHI |
| | SVAMDKFGFSLPYVQYFGGVSALSKQQFLAINGFPNNYWGWGGEDDDIFNRL |
| | VHKGMSISRPNAVVGRCRMIRHSRDKKNEPNPQRFDRIAHTKETMRFDGLNSL |
| | TYKVLDVQRYPLYTQITVDIGTPR |
| SEQ ID | Mouse B4GALT1 Variant 2 |
| NO: 25 | ATGCCGGGCGCGACCCTGCAGCGGGCCTGCCGCCTGCTCGTGGCCGTCTGC |
| | GCGCTGCACCTCGGCGTCACCCTCGTCTATTACCTCTCTGGCCGCGATCTGA |
| | GCCGCCTGCCCCAGTTGGTCGGAGTCTCCTCTACACTGCAGGGCGGCACGA |
| | ACGGCGCCGCAGCCAGCAAGCAGCCCCCAGGAGAGCAGCGGCCGCGGGGT |
| | GCGCGGCCGCCGCCTCCTTTAGGCGTCTCCCCGAAGCCTCGCCCGGGTCTCG |
| | ACTCCAGCCCTGGTGCAGCTTCTGGCCCCGGCTTGAAGAGCAACTTGTCTTC |
| | GTTGCCAGTGCCCACCACCACTGGACTGTTGTCGCTGCCAGCTTGCCCTGAG |
| | GAGTCCCCGCTGCTCGTTGGCCCCATGCTGATTGACTTTAATATTGCTGTGG |
| | ATCTGGAGCTTTTGGCAAAGAAGAACCCAGAGATAAAGACGGGCGGCCGTT |
| | ACTCCCCCAAGGACTGTGTCTCTCCTCACAAGGTGGCCATCATCATCCCATT |
| | CCGTAACCGGCAGGAGCATCTCAAATACTGGCTGTATTATTTGCATCCCATC |
| | CTTCAGCGCCAGCAACTCGACTATGGCATCTACGTCATCAATCAGGCTGGA |
| | GACACCATGTTCAATCGAGCTAAGCTGCTCAATATTGGCTTTCAAGAGGCCT |
| | TGAAGGACTATGATTACAACTGCTTTGTGTTCAGTGATGTGGACCTCATTCC |
| | GATGGACGACCGTAATGCCTACAGGTGTTTTTCGCAGCCACGGCACATTTCT |
| | GTTGCAATGGACAAGTTCGGGTTTAGCCTGCCATATGTTCAGTATTTTGGAG |
| | GTGTCTCTGCTCTCAGTAAACAACAGTTTCTTGCCATCAATGGATTCCCTAA |
| | TAATTATTGGGGTTGGGGAGGAGAAGATGACGACATTTTTAACAGATTAGT |
| | TCATAAAGGCATGTCTATATCACGTCCAAATGCTGTAGTAGGGAGGTGTCG |
| | AATGATCCGGCATTCAAGAGACAAGAAAAATGAGCCCAATCCTCAGAGGTT |
| | TGACCGGATCGCACATACAAAGGAAACGATGCGCTTCGATGGTTTGAACTC |
| | ACTTACCTACAAGGTGTTGGATGTACAGAGATACCCGTTATATACCCAAATC |
| | ACAGTGGACATCGGGACACCGAGATAG |
| SEQ ID | Mouse B4GALT1 Variant 2 |
| NO: 26 | MPGATLQRACRLLVAVCALHLGVTLVYYLSGRDLSRLPQLVGVSSTLQGGTN |
| | GAAASKQPPGEQRPRGARPPPPLGVSPKPRPGLDSSPGAASGPGLKSNLSSLPVP |
| | TTTGLLSLPACPEESPLLVGPMLIDFNIAVDLELLAKKNPEIKTGGRYSPKDCVSP |
| | HKVAIIIPFRNRQEHLKYWLYYLHPILQRQQLDYGIYVINQAGDTMFNRAKLLN |
| | IGFQEALKDYDYNCFVFSDVDLIPMDDRNAYRCFSQPRHISVAMDKFGFSLPYV |
| | QYFGGVSALSKQQFLAINGFPNNYWGWGGEDDDIFNRLVHKGMSISRPNAVV |
| | GRCRMIRHSRDKKNEPNPQRFDRIAHTKETMRFDGLNSLTYKVLDVQRYPLYT |
| | QITVDIGTPR |
| SEQ ID | Rat B4GALT1 |
| NO: 27 | ATGAGGTTTCGTGAGCCGTTCCTGGGCGGCAGCGCCGCGATGCCGGGCGCG |
| | ACCCTGCAGCGGGCCTGCCGCCTGCTCGTGGCGGTCTGCGCGCTGCACCTTG |
| | GCGTCACCCTGGTCTATTACCTCTCCGGTCGCGATCTGAGCCGCCTGCCCCA |
| | ACTGGTCGGAGTCTCCTCTTCACTGCAAGGCGGCACGAACGGCGCCGCCGC |
| | CAGCAAGCAGCCCTCGGGAGAGCTCCGGCCCCGGGGCGCGCGGCCGCCGCC |
| | TCCTTTAGGCGTCTCCCCGAAGCCTCGCCCGGGTTCTGACTCCAGCCCTGAT |
| | GCGGCTTCTGGCCCCGGCCTGAAGAGCAACTTGACTTCGGTGCCAATGCCC |
| | ACCAGCACTGGATTGTTGACTCTGCCTGCTTGCCCTGAGGAGTCCCCGCTGC |
| | TCGTTGGCCCCATGGTGATTGACTTTAATATTCCTGTGGATCTGGAGCTTTTG |
| | GCAAAGAAGAACCCAGAGATAAAGATGGGCGGCCGTTACTTCCCCAAGGA |
| | CTGTATCTCCCCTCACAAGGTGGCCATCATTATCCCATTCCGTAACCGGCAG |
| | GAGCACCTCAAATACTGGCTGTATTATTTGCATCCAGTCCTTCAGCGCCAGC |
| | AACTCGACTATGGCATCTACGTCATCAATCAGGCTGGAGACACCATGTTTAA |
| | TCGAGCTAAGCTGCTCAACGTTGGCTTTCAAGAGGCCTTGAAAGACTATGA |
| | CTACAACTGCTTTGTGTTCAGTGATGTGGACCTCATTCCAATGGATGACCAT |
| | AATGCCTACAGGTGCTTTTCACAGCCACGGCATATTTCTGTCGCAATGGACA |
| | AGTTCGGGTTTAGCCTGCCTTACGTTCAGTATTTTGGAGGTGTCTCCGCTCTC |
| | AGTAAACAACAGTTCCTTACCATCAATGGATTTCCTAATAATTACTGGGGCT |
| | GGGGAGGAGAAGATGATGACATTTTTAACAGATTAGTTCATAAAGGCATGT |
| | CTATATCACGCCCAAATGCTGTGGTAGGCAGGTGTCGCATGATCCGGCACTC |
| | AAGAGACAAGAAAAATGAGCCCAACCCTCAGAGGTTTGACCGGATCGCAC |
| | ATACAAAGGAAACGATGCGCCTTGATGGTTTGAACTCACTTACCTACCAGG |
| | TGTTGGACATACAGAGATACCCGTTATATACCAAAATCACAGTGGACATCG |
| | GGACACCAAGATAG |
| SEQ ID | Rat B4GALT1 |
| NO: 28 | MRFREPFLGGSAAMPGATLQRACRLLVAVCALHLGVTLVYYLSGRDLSRLPQL |
| | VGVSSSLQGGTNGAAASKQPSGELRPRGARPPPPLGVSPKPRPGSDSSPDAASG |
| | PGLKSNLTSVPMPTSTGLLTLPACPEESPLLVGPMVIDFNIPVDLELLAKKNPEIK |
| | MGGRYFPKDCISPHKVAIIIPFRNRQEHLKYWLYYLHPVLQRQQLDYGIYVINQ |
| | AGDTMFNRAKLLNVGFQEALKDYDYNCFVFSDVDLIPMDDHNAYRCFSQPRHI |
| | SVAMDKFGFSLPYVQYFGGVSALSKQQFLTINGFPNNYWGWGGEDDDIFNRLV |
| | HKGMSISRPNAVVGRCRMIRHSRDKKNEPNPQRFDRIAHTKETMRLDGLNSLT |
| | YQVLDIQRYPLYTKITVDIGTPR |

In some embodiments, for the methods comprising introducing a polynucleotide sequence encoding 1B4GALT1 and ST6 into the CHO cell, such polynucleotide sequence is provided in the same vector. In some embodiments, the polynucleotide sequence encoding 1B4GALT1 and the polynucleotide sequence encoding ST6 are provided in two separate vectors.

In some embodiments, the polynucleotide sequence encoding ST6 and/or the polynucleotide sequence encoding 1B4GALT1 are operably linked to a promoter.

The methods described herein further comprise culturing the CHO cell that has been modified to 1) express ST6 alone; or 2) co-express 1B4GALT1 and ST6 in a culture medium comprising an amount of manganese and galactose effective to increase the sialylation of a therapeutic protein produced by cells grown in the culture medium. In one embodiment, said amount of manganese and galactose is non-toxic to the cells, i.e., does not reduce cell viability, cell growth, or protein production. In related embodiments, the disclosure provides a culture medium comprising an amount of manganese and galactose effective to increase the sialylation of a therapeutic protein produced by cells grown in this culture medium.

In some embodiments, the sialylation of the therapeutic protein produced by the CHO cell using the methods described herein is increased by at least 5% compared to a protein produced by a CHO cell cultured in a medium that does not comprise added manganese and galactose during the cell culture. In some embodiments, the sialylation of the therapeutic protein produced by the CHO cell is increased by at least about 5%, 6%, 7%, 8%, 9%, 10%, 11%, 12%, 13%, 14%, 15%, 6, 17%, 18%, 19%, 20%, 21%, 22%, 23%, 24%, 25%, 30%, 35%, 40%, 50%, 60%, 70% or more compared to a protein produced by a CHO cell cultured in a medium that does not comprise added manganese and galactose during the cell culture.

In some embodiments, the culture medium used in the methods described herein may have trace amounts of manganese ranging from 0 ppb to 200 ppb (parts per billion) (e.g., 99 ppb in liquid formulation), and the methods described herein comprise further supplementing the medium with manganese during the cell culture period. In some embodiments, trace amounts of manganese in the culture medium is 99 or 100 ppb. In some embodiments, the methods described herein comprise adding an amount of manganese to the culture medium ranging from about 40 ppb to about 500 ppb or from about 200 ppb to about 400 ppb, or from about 100 ppb to about 300 ppb. In other exemplary embodiments, the concentration of manganese (e.g., the final concentration after the manganese-supplemented medium is added to the host cells in culture) at the lower end of the desired range may range from about 40 ppb, 50 ppb, 60 ppb, 70 ppb, 80 ppb, 90 ppb, 100 ppb, 120 ppb, 140 ppb, 160 ppb, 180 ppb, 200 ppb, 220 ppb, 240 ppb, 260 ppb, 280 ppb, 300 ppb, 320 ppb, 340 ppb, 360 ppb, 380 ppb, 400 ppb, or higher; the concentration of manganese (e.g., the final concentration after the manganese-supplemented medium is added to the host cells in culture) at the higher end of the range may also range up to about 500 ppb, 450 ppb, 400 ppb, 350 ppb, 300 ppb, 280 ppb, 260 ppb, 240 ppb, 220 ppb, 200 ppb, 200 ppb, 180 ppb, 160 ppb, 140 ppb, 120 ppb, 100 ppb, 90 ppb, 80 ppb, 70 ppb, 60 ppb, 50 ppb, or 40 ppb. In some embodiments, the concentration of manganese in the culture medium (e.g., the final concentration after the manganese-supplemented medium is added to the host cells in culture) is about 300 ppb. In some embodiments, the concentration of manganese in the culture medium (e.g., the final concentration after the manganese-supplemented medium is added to the host cells in culture) is about 400 ppb. In some embodiments, the manganese is added into the medium as a manganese salt (e.g., manganese (II) sulfate, monohydrate).

In some embodiments, the culture medium used in the methods described herein may have trace amounts of galactose in standard medium compositions ranging from 0 mM to 50 mM, and the methods described herein comprise further adding an amount of galactose during the cell culture period. In some embodiments, the medium does not contain any galactose prior to the cell culture. In exemplary embodiments, the methods comprise adding galactose to the culture medium in an amount ranging from about 10 mM to about 100 mM, from about 30 mM to about 90 mM, or from about 25 mM to about 50 mM. In other exemplary embodiments, the concentration of galactose (e.g., the final concentration after the galactose-supplemented medium is added to the host cells in culture) is at the lower end of the desired range may range from about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM or higher. In some embodiments, the concentration of galactose (e.g., the final concentration after the galactose-supplemented medium is added to the host cells in culture) is at the higher end of the range may also range up to about 100 mM, 95 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, or 10 mM. In some embodiments, the concentration of galactose in the culture medium (e.g., the final concentration after the galactose-supplemented medium is added to the host cells in culture) is about 45 mM or 46 mM. In some embodiments, the concentration of galactose in the culture medium (e.g., the final concentration after the galactose-supplemented medium is added to the host cells in culture) is about 40 mM. In some embodiments, the galactose is added into the medium as galactose anhydrous.

In some embodiments, the method described herein comprises adding manganese and galactose to the medium as part of a nutrient feed at one or more timepoints during the culture period. For example, in some embodiments, the method comprises adding manganese and galactose to the medium on day 3 of the culture period. In some embodiments, the method comprises adding manganese and galactose to the medium on days 3 and 6 of the culture period. In some embodiments, the method comprises adding manganese and galactose to the medium on days 3, 6 and 8 of the culture period.

In some embodiments, the method described herein comprises adding at least 100 ppb manganese and at least 15 mM galactose to the medium as part of a nutrient feed at one or more timepoints during the culture period. For example, in some embodiments, 100 ppb manganese and 15 mM galactose are added to the medium on day 3 of the culture period. In some embodiments, the method described herein comprises adding 100 ppb manganese and 15 mM galactose to the medium on each of days 3 and 6 of the culture period. In some embodiments, the method described herein comprises adding 100 ppb manganese and 15 mM galactose to the medium on each of days 3, 6 and 8 of the culture period.

In some embodiments, the culture medium used in the methods described herein may have trace amounts of copper ranging from 0 ppb to 100 ppb (e.g., 28 ppb), and the methods described herein comprise further supplementing the medium with copper during the cell culture period. In some embodiments, the method described herein comprises adding copper to the medium, optionally in an amount ranging from about 0.01 mM to about 0.5 mM. In exemplary embodiments, the concentration of copper in the culture medium (e.g., the final concentration after the galactose and manganese-supplemented medium is added to the host cells in culture) ranges from about 0.01 mM to about 0.1 mM or from about 0.05 mM to about 0.15 mM, or from about 0.1 mM to about 0.5 mM. In some embodiments, the concentration of copper in the medium is 0.1 mM. In some embodiments, the copper stock solution can be prepared using copper (II) sulfate pentahydrate salt.

The culture medium can also include any other necessary or desirable ingredients known in the art, such as carbohydrates, including glucose, essential and/or non-essential amino acids, lipids and lipid precursors, nucleic acid precursors, vitamins, inorganic salts, trace elements including rare metals, and/or cell growth factors. The culture medium may be chemically defined or may include serum, plant hydrolysates, or other derived substances. It is contemplated that the culture medium may be essentially or entirely animal-component free. The culture medium may be essentially or entirely serum-free, protein-free, growth factor-free and/or peptone-free. Essentially serum-free means that the medium lacks any serum or contains an insignificant amount of serum. Essentially protein-free means that the medium lacks any protein or contains an insignificant amount of protein. Essentially growth factor-free means that the medium lacks any growth-factor or contains an insignificant amount of growth factor. Essentially peptone-free means that the medium lacks any peptone or contains an insignificant amount of peptone.

The culture medium may also include supplementary amino acids depleted during cell culture, e.g., asparagine, aspartic acid, cysteine, cystine, isoleucine, leucine, tryptophan, and valine. The amino acid supplementation may be in the initial growth medium and/or in medium added during or after the rapid growth phase.

The medium may include lipids and/or lipid precursors such as choline, ethanolamine, or phosphoethanolamine, cholesterol, fatty acids such as oleic acid, linoleic acid, linolenic acid, methyl esters, D-alpha-tocopherol, e.g., in acetate form, stearic acid, myristic acid, palmitic acid, palmitoleic acid, or arachidonic acid. A number of commercially available lipid mixtures are available.

The medium may include an iron supplement comprising iron and a synthetic transport molecule to which the iron binds. The medium may include inorganic compounds or trace elements, supplied as appropriate salts, such as sodium, calcium, potassium, magnesium, copper, iron, zinc, selenium, molybdenum, vanadium, manganese, nickel, silicon, tin, aluminum, barium, cadmium, chromium, cobalt, germanium, potassium, silver, rubidium, zirconium, fluoride, bromide, iodide, and chloride. A number of commercially available mixtures of trace elements are available.

The medium may also optionally include a nonionic surfactant or surface-active agent to protect the cells from the mixing or aeration. The culture medium may also comprise buffers such as sodium bicarbonate, monobasic and dibasic phosphates, HEPES, and/or Tris.

The culture medium may also comprise inducers of protein production, such as sodium butyrate, or caffeine. Other known inducers include, but are not limited to, the following compounds: N-Acetyl-L-cysteine, Actinomycin D, 7-Amino-, Bafilamycin A1, Streptomyces griseus, Calphostin C, Cladosporium cladosporioides, Camptothecin, Camptotheca acuminata, CAPE, 2-Chloro-2′-deoxyadenosine, 2-Chloro-2′-deoxyadenosine 5′-Triphosphate, Tetralithium Salt, Cycloheximide, Cyclophosphamide Monohydrate, Cyclosporine, Trichoderma polysporum, Daunorubicin, Hydrochloride, Dexamethasone, Doxorubicin, Hydrochloride, (−)-Epigallocatechin Gallate, Etoposide, Etoposide Phosphate, ET-18-OCH3, 5-Fluorouracil, H-7, Dihydrochloride, Genistein, 4-Hydroxynonenal, 4-Hydroxyphenylretinamide, Hydroxyurea, IL-1β Inhibitor, (±)-S-Nitroso-N-acetylpenicillamine, S-Nitrosoglutathione, Phorbol-12-myristate-13-acetate, Puromycin, Dihydrochloride, 1-Pyrrolidinecarbodithioic Acid, Ammonium Salt, Quercetin, Dihydrate, Rapamycin, Sodium Butyrate, Sodium 4-Phenylbutyrate, D-erythro-Sphingosine, N-Acetyl-, D-erythro-Sphingosine, N-Octanoyl-, Staurosporine, Streptomyces sp., Sulindac, Thapsigargin, TRAIL, E. coli, Trichostatin A, Streptomyces sp., (±)-Verapamil, Hydrochloride, Veratridine, Vitamin D3, and Vitamin E Succinate (VWR and Calbiochem).

The culture medium optionally excludes A23187 or other compounds which deplete divalent cations.

### Maintaining Cells in a Cell Culture

With regard to the methods of therapeutic protein composition of the present disclosure, the therapeutic protein composition can be produced by maintaining cells in a cell culture. The cell culture can be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 1×10⁶ cells/mL in 1.5 L medium.

In exemplary aspects, the methods of the disclosure comprise maintaining the cells in a cell culture medium at a pH of about 6.5 to about 7.2, e.g., in various aspects, about 6.5, about 6.55, about 6.6, about 6.65, about 6.7, about 6.75, about 6.8, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.0, about 7.02, about 7.03, about 7.04, about 7.05, about 7.06, about 7.07, about 7.08, about 7.09, about 7.1, about 7.11, about 7.12, about, about 7.13, about 7.14, about 7.15, about 7.16, about 7.17, about 7.18, about 7.19 or about 7.2.

In some embodiments, the method comprises maintaining the cell culture at a temperature between 30° C. and 40° C. In exemplary embodiments, the temperature is between about 32° C. to about 38° C. or between about 35° C. to about 38° C.

In some embodiments, the method comprises one or more temperature and/or pH shifts during a cell culture. These shifts can be used, for example, to influence the behavior of the cells. Higher temperatures are typically used to encourage cell growth, lower temperatures are typically used to slow growth and encourage production of recombinant protein.

In some embodiments, the method comprises maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, about 225, about 250, about 275, about 300, about 325, about 350, about 375, about 400, about 425, about 450, about 475, or about 500.

In some embodiments, the method comprises maintaining a dissolved oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

In some embodiments, the cell culture is maintained in a medium suitable for cell growth and/or is provided with one or more feeding medium according to any suitable feeding schedule.

In some embodiments, the type of cell culture is a fed-batch culture or a continuous perfusion culture. However, the methods of the disclosure are advantageously not limited to any particular type of cell culture.

Methods for controlling sialylation of a recombinant glycoprotein, particularly for controlling N-glycolylneuraminic acid (NGNA) levels in the sugar chains, are described in U.S. Pat. No. 5,459,031, incorporated herein by reference in its entirety, and such methods may be used in conjunction with the culture medium and culture methods described herein. The methods involve adjusting culture parameters, including the carbon dioxide level, to achieve the desired NGNA content in carbohydrate.

### Evaluation of Glycosylation and Sialylation

Recombinant glycoproteins produced in CHO cells can exhibit variable glycosylation and sialylation. Highly sialylated forms of glycoprotein molecules can be separated from lower sialylated (including non-sialylated) forms of such molecules via anion exchange chromatography. Sialic acids, being acidic and thus negatively charged, are captured on the column, so that highly sialylated molecules are retained on the column while lower sialylated forms flow through. The amount of glycoprotein in each fraction (retained on column vs. flow through fraction) can be determined and compared to the starting amount of glycoprotein loaded from the cell culture medium.

In some embodiments, the sialic acid described herein is α2,6-sialylated glycan. In some embodiments, the level of α2,6-sialylated glycans is kept constant in the protein produced with extended cell culture duration. In some embodiments, the extended cell culture duration is about 1 population doubling (PDL), 2 PDL, 3 PDL, 4 PDL, 5 PDL, 6 PDL, 7 PDL, 8 PDL, 9 PDL, 10 PDL, 11 PDL, 12 PDL, 13 PDL, 14 PDL, 15 PDL, 16 PDL, 17 PDL, 18 PDL, 19 PDL, 20 PDL, 21 PDL, 22 PDL, 23 PDL, 24 PDL, 25 PDL, 26 PDL, 27 PDL, 28 PDL, 29 PDL, 30 PDL, 31 PDL, 32 PDL, 33 PDL, 34 PDL, 35 PDL, 36 PDL, 37 PDL, 38 PDL, 39 PDL, 40 PDL, 41 PDL, 42 PDL, 43 PDL, 44 PDL, 45 PDL, 46 PDL, 47 PDL, 48 PDL, 49 PDL, 50 PDL, 55 PDL, 60 PDL, 65 PDL, 70 PDL, 75 PDL, 80 PDL, 85 PDL, 90 PDL, 95 PDL, or 100 PDL. In some embodiments, the extended cell culture duration is about 27 PDL.

For glycoprotein compositions, an increase or improvement in sialylation can be determined by anion exchange chromatography according to Elliott et al., Biochemistry, 33(37):11237-45 (1994), herein incorporated by reference in its entirety. More highly sialylated proteins are expected to be more negatively charged and bind more strongly to the column, while less sialylated proteins flow through or are easily eluted. The amount of glycoprotein molecules in each of the two fractions (retained on resin vs. flow through fraction) can be determined, e.g., by ELISA, and compared to the starting amount of such molecules loaded from the cell culture medium. Exemplary ELISA kits are sold commercially and include R & D Systems, IVD Human EPO EIA kit.

Chromatography is carried out as follows. To eliminate cells and debris, medium in which mammalian cells that produce therapeutic protein, have been cultured is centrifuged at about 1000 rpm and filtered through a 0.45 micron filter. In an alternate method, to remove cells and debris, medium in which mammalian cells that produce therapeutic protein, is filtered using depth filtration. The filtered material is then subjected to anion exchange chromatography in order to pre-purify a fraction containing primarily the four to seven most highly sialylated species of the glycoprotein molecules. A strong ion exchange resin may be used, such as, for example, TRICORN™ Mono-Q 5/50 GL (Amersham, part #17-5166-01) or other strong anion exchange resins, particularly those that have the quaternary amine —CH₂-N+-(CH₃)₃ as the functional group of the resin. The exact procedure will depend on the theoretical maximum number of sialic acid residues that the particular glycoprotein molecules can contain. The buffers used to elute the glycoprotein molecules from the anion exchange column are designed to: (1) elute from the column most or all protein molecules belonging to species that are less sialylated than a group of species consisting of approximately the top third most highly sialylated species (the “highly sialylated” species are those having greater than or equal to 3 terminal sialic acid residues per protein molecule (2) then elute protein molecules belonging to the four to seven most highly sialylated species, and (3) finally remove more highly charged species from the column, which may include glycoforms bearing sulfated N-glycans. Therefore, the exact composition of the wash and elution buffers can be adjusted according to the theoretical maximum number of sialic acid residues on the glycoprotein molecule. One of skill in the art can make such adjustments based on routine empirical optimization of column parameters and assaying the material coming off the column on analytical isoelectric focusing gels.

An increase in the percentage of antigen-binding proteins recovered from the pool retained on the resin (or a reduction in the percentage of such molecules observed in the flow through fraction) relative to the control (e.g. produced from medium with no manganese or trace element amounts of manganese) indicates an increase in sialylation, whether through increasing the percentage of sialylated molecules produced or through increasing their degree of sialylation.

The actual glycan structure can be determined by any techniques known in the art, including enzymatic digestion of carbohydrate, lectin immunoblotting, 1D and 2D 1H-NMR spectroscopy, mass spectroscopy techniques including electrospray ionization tandem mass spectrometry (ESI MS) or matrix assisted laser desorption ionization time-of-flight mass spectrometry (MALDI-TOF MS), and/or fluorescent labeling of enzymatically released N-glycans followed by resolution by HPLC and comparison to known N-glycan control samples.

An exemplary technique, described in the examples below, for determining the amount of glycoprotein with an occupied O-glycosylation site involves N-Glycanase digestion to remove the N-linked carbohydrates followed by reverse phase-HPLC to separate the glycoprotein composition into two peaks. Peak identification as occupied O-site or unoccupied O-site can be confirmed by mass spectrometry.

N-site branching and sialylation, including the percentage of sialylated molecules produced and the degree of sialylation of the sialyated molecules, can be determined by analyzing the glycoproteins for structural content by N-glycan mapping and enzymatic sequencing, e.g. by digestion with N-Glycanase and neuraminidase, coupled with MALDI-TOF mass spectrometry for size determination of the released sugars. An exemplary technique is described in the examples below.

The percent of the sugars attached to the antigen-binding proteins that are galactose can be determined, e.g., by neuraminidase plus galactosidase digestion followed by HPLC separation or MALDI-TOF mass spectrometry for size determination of the released sugars. An exemplary technique is described in the examples below.

### Therapeutic Proteins

In various aspects, the therapeutic protein is an antibody protein product. As used herein, the term “antibody protein product” refers to any one of several antibody alternatives which in various instances is based on the architecture of an antibody but is not found in nature. In some aspects, the antibody protein product has a molecular-weight within the range of at least about 12-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of
15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the therapeutic protein is a bispecific T cell engager (BiTE®) molecule, which is an artificial bispecific monoclonal antibody. Canonical BiTE@molecules are fusion proteins comprising two scFvs of different antibodies. One binds to CD3 and the other binds to a target antigen. BiTE® molecules are known in the art. See, e.g., Huehls et al., Immuno Cell Biol 93(3): 290-296 (2015); Rossi et al., MAbs 6(2): 381-91 (2014); Ross et al., PLoS One 12(8): e0183390.

In exemplary aspects, the therapeutic protein is a chimeric antigen receptor (CAR). Chimeric antigen receptors are genetically engineered fusion proteins constructed from multiple domains typically of other naturally occurring molecules expressed by immune cells. In several aspects, CARs comprises an extracellular antigen-binding domain or antigen recognition domain, a signaling domain and a co-stimulatory domain. CARs are described in the art. See, e.g., Maus et al., Clin Cancer Res (2016) 22(8): 1875-1884; Dotti et al., Immuno Rev (2014) 257(1): 10.1111/imr.12131; Lee et al., Clin Cancer Res (2012): 18(10): 2780-2790; and June and Sadelain, NEJM (2018) 379: 64-73.

Antibodies are frequently glycosylated in the constant domain region. N-glycosylation sites have been detected as follows, based on numbering from the N-terminus of the heavy chain constant region: IgG1-N180; IgG2-N176; IgG3-N227; IgG4-N177; IgM-N46, N209, N272, N279, N439; IgAQ1-N144, N340; IgA2-N47, N131, N205, N327 (Chandler et al., Molecular & Cellular Proteomics 18: 686-703, 2019). It has been shown that the level of sialic acid in human endogenous IgGs is approximately 11%-15% (Boune et al., Antibodies 9: 22, 2020). In various embodiments, the sialic acid is on an N-glycosylation site described herein. In various embodiments, the sialic acid is on N180 (N297 if numbering from variable region) of IgG1. In various embodiments, the sialic acid is on a residue in the Fab region. In various embodiments, an antibody composition herein comprises at least about 20%, of the antibodies which are sialylated. In various embodiments, at least about 21%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more of the antibodies comprise at least one sialic acid residue.

Exemplary therapeutic proteins include, but are not limited to, CD proteins, growth factors, growth factor receptor proteins (e.g., HER receptor family proteins), cell adhesion molecules (for example, LFA-I, MoI, pl50, 95, VLA-4, ICAM-I, VCAM, and alpha v/beta 3 integrin), hormone (e.g., insulin), coagulation factors, coagulation-related proteins, colony stimulating factors and receptors thereof, other receptors and receptor-associated proteins or ligands of these receptors, and viral antigens.

Exemplary therapeutic proteins include, e.g., any one of the CD proteins, such as CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79u, CD790, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

Exemplary growth factors, include, for instance, vascular endothelial growth factor (“VEGF”), growth hormone, thyroid stimulating hormone (TSH), follicle stimulating hormone (FSH), luteinizing hormone (LH), growth hormone releasing factor (GHRF), parathyroid hormone (PTH), Mullerian-inhibiting substance (MIS), human macrophage inflammatory protein (MIP-I-alpha), erythropoietin (EPO), nerve growth factor (NGF), such as NGF-beta, platelet-derived growth factor (PDGF), fibroblast growth factors (FGF), including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors. The therapeutic protein in some aspects is an insulin or insulin-related protein, e.g., insulin, insulin A-chain, insulin B-chain, proinsulin, and insulin-like growth factor binding proteins. Exemplary growth factor receptors include any receptor of any of the above growth factors. In various aspects, the growth factor receptor is a HER receptor family protein (for example, HER2, HER3, HER4, and the EGF receptor), a VEGF receptor, TSH receptor, FSH receptor, LH receptor, GHRF receptor, PTH receptor, MIS receptor, MIP-1-alpha receptor, EPO receptor, NGF receptor, PDGF receptor, FGF receptor, EGF receptor, (EGFR), TGF receptor, or insulin receptor.

Exemplary coagulation and coagulation-related proteins, include, for instance, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fins). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-I to IL-33 and IL-I to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Additional exemplary therapeutic proteins include, e.g., myostatins, TALL proteins, including TALL-I, amyloid proteins, including but not limited to amyloid-beta proteins, thymic stromal lymphopoietins (“TSLP”), RANK ligand (“OPGL”), c-kit, TNF receptors, including TNF Receptor Type 1, TRAIL-R2, angiopoietins, and biologically active fragments or analogs or variants of any of the foregoing.

In exemplary aspects, the therapeutic protein is any one of the pharmaceutical agents known as Activase® (Alteplase); alirocumab, Aranesp® (Darbepoetin-alfa), Epogen® (Epoetin alfa, or erythropoietin); Avonex® (Interferon β-Ia); Bexxar® (Tositumomab); Betaseron® (Interferon-β); bococizumab (anti-PCSK9 monoclonal antibody designated as L1L3, see U.S. Pat. No. 8,080,243); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoetin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMl); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara® (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-MRSA mAb), IL-I Trap (the Fc portion of human IgG1 and the extracellular domains of both IL-I receptor components (the Type I receptor and receptor accessory protein)), VEGF Trap (Ig domains of VEGFR1 fused to IgG1 Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi® (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-sclerostin antibodies (see, U.S. Pat. No. 8,715,663 or U.S. Pat. No. 7,592,429) anti-sclerostin antibody designated as Ab-5 (U.S. Pat. No. 8,715,663 or U.S. Pat. No. 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AlN457); anti-IL2Ra mAb (HuMax-TAC); anti-IL5 Receptor mAb; anti-integrin receptors mAb (MDX-018, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDlmAb (MDX-1 106 (ONO-4538)); anti-PDGFRu antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; NVS Antibody #2; or an amyloid-beta monoclonal antibody.

In some embodiments, the antibody is an anti-IL12 antibody, anti-IL-23 antibody, or an anti-IL12/23 antibody (e.g., ustekinumab antibody comprising the heavy chain sequence according to SEQ ID NO: 29 and the light chain sequence according to SEQ ID NO: 30).

In some embodiments, the therapeutic polypeptide is a BiTE® molecule. Blinatumomab (BLINCYTO®) is an example of a BiTE® molecule, specific for CD19. BiTE® molecules that are modified, such as those modified to extend their half-lives, can also be used in the disclosed methods.

In exemplary aspects, the level of ADCC of an antibody composition is determined by a quantitative cell-based assay which measures the ability of the antibodies of the antibody composition to mediate cell cytotoxicity in a dose-dependent manner in cells expressing the antigen of the antibodies and engaging Fc-gammaRIIIA receptors on effector cells through the Fc domain of the antibodies. In various embodiments, the method comprises the use of target cells harboring detectable labels that are released when the target cells are lysed by the effector cells. The amount of detectable label released from the target cells is a measure of the ADCC activity of the antibody composition. The amount of detectable label released from the target cells in some aspects is compared to a baseline. Also, the ADCC level may be reported as a % ADCC relative to a control % ADCC. In various aspects, the % ADCC is a relative % ADCC, which optionally, is relative to a control % ADCC. In various aspects, the control % ADCC is the % ADCC of a reference antibody.

In some embodiments, the therapeutic protein (e.g., antibody) produced according to the method described herein can be used in treating patients with plaque psoriasis, psoriatic arthritis, Crohn's disease, and/or ulcerative colitis. For example, patients with moderate to severe plaque psoriasis can be treated with an anti-IL12/23 antibody produced by the method described herein. Further, patients with moderate to severely active Crohn's disease or ulcerative colitis can be treated with an anti-IL12/23 antibody produced by the method described herein. Further, patients with active psoriatic arthritis can be treated, alone or in combination with methotrexate, with an anti-IL12/23 antibody produced by the method described herein.

All patents and other publications identified are expressly incorporated herein by reference in their entirety or in relevant part, as would be apparent from the context of the citation, for the purpose of describing and disclosing, for example, the methodologies described in such publications that might be used in connection with information described herein.

The following examples are given merely to illustrate the present invention and not in any way to limit its scope.

### EXAMPLES

### Example 1—Overexpression of ST6 or of Both ST6 and B4GALT1 in a CHO Cell Increases Sialylation of a Therapeutic Protein

This example demonstrates that CHO cells overexpressed with either ST6 (α2,6-sialyltransferase-1) alone or both ST6 and β1,4-galactosyltransferase 1 (B4GALT1) increased the sialyation content of a recombinant anti-IL12/23 antibody (i.e., ustekinumab antibody comprising the heavy chain sequence according to SEQ ID NO: 29 and the light chain sequence according to SEQ ID NO: 30).

Eleven (11) different amplified CHO (Chinese Hamster Ovary) CS9 cell line (e.g., a DHFR (dihydrofolate reductase) deficient cells derived from DXB-11 cells) pools expressing a recombinant anti-IL12/23 antibody were transfected with ST6 (SEQ ID NO: 1) or both ST6 and B4GALT1 (SEQ ID NO: 13) and resulted in 33 engineered pools. As the focus was to increase the α2,6 linkage and percent N-Acetylneuraminic Acid (% NANA), the expression of α-2,6 linkage by flow cytometry using SNA lectins was assessed. Lectins are proteins that bind to the specific glycol/sugar portion of glycans. Sambucus nigra lectin (SNA1), isolated from elderberry bark, binds preferentially to sialic acid attached to terminal galactose in α-2,6 linkage. Analysis showed the different engineered pools exhibited a 3.3%-25% positive population for SNA i.e., α-2,6 linkage. The SNA positive population was enriched using FACS (Fluorescence-Activated Cell Sorting) sorted using a lectin-based cell surface qualitative FACS assay.

Cloning was carried out both by FACS and Beacon (BLI) platform. During FACS cloning, a surface staining protocol using SNA-FITC, was used. The 386 clones derived from FACS cloning were narrowed down to 96 clones by criteria that included % SNA FITC population, Fortebio titer and a Quantigene mRNA assay that measured mRNA levels of ST6 and B4GALT1. In addition, 12 Beacon derived clones were also obtained based on positive SNA-bead assay, titer estimation by Spotlight, and growth upon expansion and transfer from the pen.

The clones were passaged and screened in a day 24 deep well plate fed-batch. The closes were screened for those having a titer >0.5 mg/ml and % SA level between 8-73% (as measured by the fast glycan method (Shah et al., “Rapid Automated LC-MS/MS Glycan Analysis for Monoclonal Antibodies”, American Society of Mass Spectrometry (ASMS) conference (Jun. 4-9, 2016)), and 34 clones were selected for further analysis. These 34 clones were analyzed for their glycan profile using Hydrophilic Interaction Liquid. Chromatography (HILIC). The 34 clones were narrowed down to 16 by eliminating clones with >10% high mannose (HM). Of the sixteen clones, eight were selected for further screening based on pool diversity, low % HM, and a range of percent sialic acid (% SA). Five of the eight clones had percent sialic acid ranging from 16.7% to 27% SA.

### Example 2—Overexpression of ST6 or of Both ST6 and B4GALT1 in CHO Cells Maintain Genetic Stability Throughout the Production Process

A pre-Master Cell Bank (pre-MCB) stage stability study was performed on 3 clones: clone 1 (ST6 overexpression), clone 2 (ST6 and B4GALT1 overexpression), and clone 3 (ST6 overexpression) over defined duration and intervals of Population Doubling Levels (PDLs). The primary purpose of this study was to detect changes in growth rates, expression titers, and glycans produced as a function of cell age using methods that mimic the cell banking and manufacturing process. Additional product quality change as a function of cell age were assessed by monitoring the high molecular weight (HMW).

The pre-master cell bank (Pre-MCB) was the earliest tested/characterized cell bank of the final recombinant clone, and was used for creation of the master cell bank (MCB). Pre-MCB was used as starting material to create four additional test “mock” banks, with each bank representing defined PDL accumulation, representing different manufacturing. See Table 2. For the purposes of this study, “0 PDL” has been assigned to the PDL of the pre-MCB.

TABLE 2

| Test Banks | PDL | Represents |
|---|---|---|
| Pre-MCB | 0 | |
| Mock MCB | 15 | MCB |
| Mock WCB | 30 | WCB |
| Mock WCB + 20 PDLs | 50 | WCB + LIVCA requirement |

“Mock” cell banks that mimic the PDL levels representing MCB were created at 10-12 PDLs from the pre-MCB. A mock WCB was then created by thawing a vial of the mock MCB and accumulating an additional ~15-17 PDLs. Additionally, the “Mock” WCB was thawed and cultured to accumulate the appropriate amount of PDLs to create a “Mock” WCB+20 PDLs bank and a “Mock” WCB+35 PDLs bank.

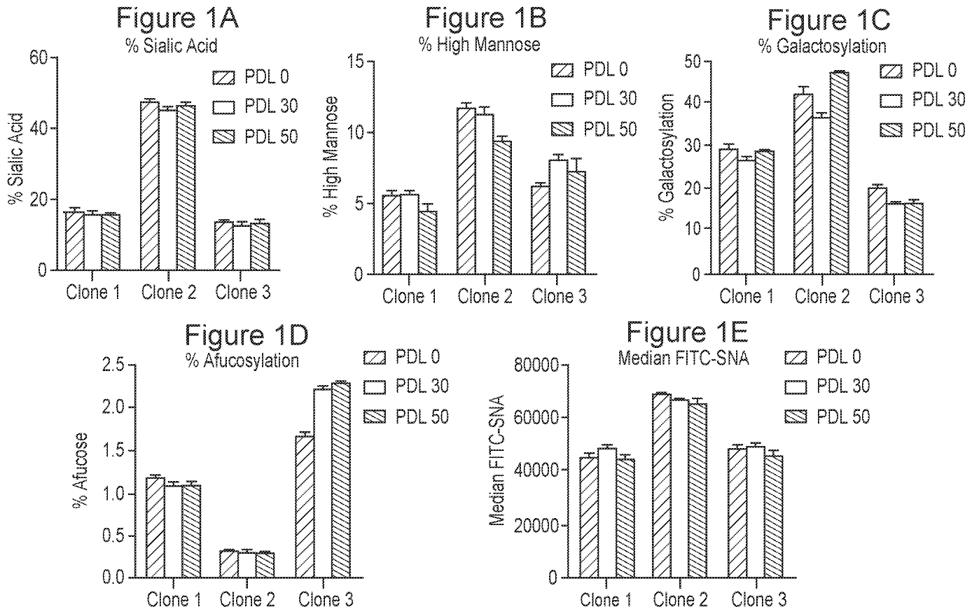

The top clone (no. 1) exhibited stable growth and productivity for OPDL, 30PDL and 50PDL samples with only 9% CV for titer (coefficient of variance=std deviation/mean), whereas clone nos. 2 and 3 had % CV of 13% and 64%, respectively. Acceptable HMW was seen in all three clones with PDL but SEC profile of clone 2 showed increase in % LMW with PDL related to the extra peak seen past LC in rCE-SDS. In terms of glycans, % sialic acid, % high mannose, % beta galactose and % afucose were observed. All three clones showed a stable glycan profile across PDLs (See FIG. 1). Similarly, all three clones maintained high FITC Signal Intensity (10⁴-10⁵) through 0, 30, and 50 PDL, with % CV of less than 1%, suggesting consistent expression of α-2,6 linkage NANA glycan mediated by the ST6 enzyme (See FIG. 1).

The genetic characterization of the mock stability samples included a copy number analysis of HC (Heavy Chain), LC (Light chain), ST6 and B4GALT1 using qPCR technique, the clones were evaluated with and without puromycin selection. Clone no. 2 and clone no. 1 showed consistent expression of all 4 attributes, whereas clone no. 3 showed loss in copy number for HC and LC, which is consistent with the titer reduction observed with clone no. 3 in fed batch.

Later, several assays were used to confirm the genetic stability of the antibody cell line based on clone no. 1 from the thaw of the MCB through the creation of the working cell bank (WCB) to end of production (EOP) at the limit of in-vitro cell age (LIVCA). The complementary deoxyribonucleic acid (cDNA) for the anti-IL12/23 antibody LC and HC was prepared by reverse transcription-polymerase chain reaction (RT-PCR), and subsequent sequence analysis demonstrated that the nucleotide sequences of the antibody LIVCA bank were identical to the nucleotide sequences in the MCB, WCB, and EOP from a typical production run. The sequences obtained also confirmed a match to the expected cDNA sequences for the antibody. Digital droplet PCR (ddPCR) was used to determine the genomic DNA (gDNA) copy number of the antibody LC and HC genes integrated in the cell line. Copy numbers in EOP from a typical production and LIVCA samples were similar to MCB and WCB. Southern blot analysis for each of the cell bank samples showed that the product gene coding for the antibody LC and HC integrated into the host genomic DNA (gDNA) were stable. A stable rearrangement was observed for each the antibody LC and antibody HC gene. Southern blot integration site analysis of the MCB, WCB, EOP from a typical production run, and LIVCA samples showed similar banding patterns indicating integration site genetic stability. Northern blot analysis showed that the antibody LC and HC expected transcripts were present and stable in each of the cell bank samples. Genetic characterization assays were also performed to characterize the stability of the human ST6 gene in the cell banks. Analysis of ST6 gene in the antibody cell banks showed that the nucleotide sequences of the LIVCA bank were identical to the nucleotide sequences in the MCB, WCB, and EOP from a typical production run. The ST6 gene also had similar copy number across all the cell banks. Additionally, the expected transcript was present and stable in each of the cell bank.

The results from these assays indicate that the antibody cell line ST6 gene or both ST6 and B4GALT1 maintains genetic stability.

### Example 3—Manganese and Galactose as CHO Cell Culture Components Increase Sialylation of a Therapeutic Protein

The following Example describes an upstream fed batch process useful to produce a recombinant anti-IL12/23 antibody in a CHO cell (by the methods described in Example 1).

Total galactose concentration, added as a separate stock solution or as part of the basal and feed medium (which has zero galactose prior to the addition), was studied in combination with production bioreactor pH to modulate sialyation of the recombinant protein. Based on the earlier studies, 100 ppb manganese (Mn) was added to the basal and feed medium (which has about 99 ppb of baseline levels of Mn) leading to a total addition of 400 ppb of Mn to the reactor over the entire duration of the production stage. 40 mM cumulative galactose was studied at pH 6.95 and 7.10, and it was observed that increasing pH increased sialyation. The basal medium volume target was 1,175L, and the feed medium volume total was 400L for days 3, 6, and 8. Accordingly, throughout the production, a total of 1) 2.115 g manganese salt (e.g., manganese (II) sulfate, monohydrate was added via basal medium at 357 mg and via feeds at 1758 mg.), and 2) 15.4 kg of galactose anhydrous was added.

Further, 20 mM, 40 mM and 60 mM galactose were studied at a pH set point of 7.10. Additionally, 40 mM galactose was also studied at pH of 7.00. A final study with a 46 mM galactose concentration was performed at pH of 7.00. Based on the data presented in the example, about 46.08 mM galactose final and 400 ppb of Mn at a pH setpoint of 7.00 resulted in sialic acid levels at least above 22%.

What is claimed is:

1. A method of increasing sialic acid content of a therapeutic protein produced by a Chinese Hamster Ovary (CHO) cell comprising expressing an a2,6-sialyltransferase-1 (ST6) (SEQ ID NO: 1, 3, 5, 7, 9, or 11) in the CHO cell.

2. A method of increasing sialic acid content of a therapeutic protein produced by a Chinese Hamster Ovary (CHO) cell comprising co-expressing an a2,6-sialyltransferase-1 (ST6) (SEQ ID NO: 1, 3, 5, 7, 9, or 11) and a b1,4-galactosyltransferase 1 (B4GALT1) (SEQ ID NO: 13, 15, 17, 19, 21, 23, 25, or 27) in the CHO cell.

3. The method of claim 1 or 2, wherein the sialic acid is α2,6-sialylated glycan.

4. The method of any one of claim 1, wherein the protein is an antibody or antigen-binding fragment thereof, a derivative of an antibody or antibody fragment, a bi-specific T-cell engager molecule, or a fusion polypeptide.

5. The method of claim 4, wherein the antibody is an anti-IL12 antibody, anti-IL23 antibody, or an anti-IL12/23 antibody.

6. A method of increasing sialic acid content of a therapeutic protein produced by a Chinese Hamster Ovary (CHO) cell comprising:
(a) expressing an α2,6-sialyltransferase 1 (ST6) (SEQ ID NO: 1, 3, 5, 7, 9, or 11) in the CHO cell; and
(b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

7. A method of increasing sialic acid content of a therapeutic protein produced by a Chinese Hamster Ovary (CHO) cell comprising:
(a) expressing an a2,6-sialyltransferase-1 (ST6) (SEQ ID NO: 1, 3, 5, 7, 9, or 11) and a b1,4-galactosyltransferase 1 (B4GALT1) (SEQ ID NO: 13, 15, 17, 19, 21, 23, 25, or 27) in the CHO cell; and
(b) culturing the cell in a medium comprising galactose and manganese, wherein culturing the cell in the medium increases sialylation of the protein produced by the cell compared to a CHO cell cultured in a medium that does not comprise added manganese and galactose during the cell culture.

8. The method of claim 6, wherein the sialylation of the protein produced by the CHO cell is increased by at least 10% compared to a protein produced by a CHO cell cultured in a medium that does not comprise added manganese and galactose during the cell culture.

9. The method of claim 6, wherein the sialylation of the protein produced by the CHO cell is increased by at least 20% compared to a protein produced by a CHO cell cultured in a medium that does not comprise added manganese and galactose during the cell culture.

10. The method of any one of claim 6, comprising adding manganese and galactose to the medium on day 3 of the cell culture.

11. The method of claim 10, wherein at least 100 ppb (Parts Per Billion) manganese and at least 15 mM galactose is added to the medium on day 3.

12. The method of any one of claim 6, further comprising adding manganese and galactose to the medium on day 6.

13. The method of claim 12, wherein at least 100 ppb manganese and at least 15 mM galactose is added to the medium on day 6.

14. The method of any one of claim 6, further comprising adding manganese and galactose to the medium on day 8.

15. The method of any one of claim 6, wherein the medium further comprises copper.

16. The method of any one of claim 6, comprising adding about 10 mM to about 100 mM galactose cumulatively over the culture period.

17. The method of claim 16, comprising adding about 45 mM galactose cumulatively over the culture period.

18. The method of any one of claim 6, comprising adding about 40 ppb to about 400 ppb manganese cumulatively over the culture period.

19. The method of any one of claim 6, comprising adding about 400 ppb manganese cumulatively over the culture period.

20. The method of any one of claim 15, comprising adding about 0.01 mM to about 0.5 mM copper cumulatively over the culture period.

21. The method of claim 20, comprising adding about 0.1 mM copper cumulatively over the culture period.

22. The method of any one of claim 6, wherein the protein is a secreted and recombinant protein.

23. The method of any one of claim 6, wherein the protein is an antibody or antigen-binding fragment thereof, a derivative of an antibody or antibody fragment, a bi-specific T-cell engager molecule, or a fusion polypeptide.

24. The method of claim 23, wherein the antibody is an anti-IL12 antibody, an anti-IL23 antibody, or an anti-IL12/23 antibody.

25. The method of any one of claim 6, wherein the sialic acid is α2,6-sialylated glycan.

26. The method of claim 25, wherein the level of α2,6-sialylated glycan is confirmed by hydrophilic interaction liquid chromatography (HILIC)-mass spectrometry (MS) analysis.

27. The method of claim 25, wherein the level of α2,6-sialylated glycans is kept constant in the protein produced with extended cell culture duration.

28. The method of claim 27, wherein the extended cell culture duration is about 27 population doublings (PDL).


SEQUENCE LISTING (30 sequences)

SEQ ID NO: 1 - DNA, 1221 bp, Homo sapiens
  source 1..1221: mol_type = other DNA
atgattcacaccaacctgaagaaaaagttcagctgctgcgtcctggtctttcttctgtttgcagtcatctgtgtgtggaaggaaaagaagaaagggagttactatgattcctttaaattgcaaaccaaggaattccaggtgttaaagagtctggggaaattggccatggggtctgattcccagtctgtatcctcaagcagcacccaggacccccacaggggccgccagaccctcggcagtctcagaggcctagccaaggccaaaccagaggcctccttccaggtgtggaacaaggacagctcttccaaaaaccttatccctaggctgcaaaagatctggaagaattacctaagcatgaacaagtacaaagtgtcctacaaggggccaggaccaggcatcaagttcagtgcagaggccctgcgctgccacctccgggaccatgtgaatgtatccatggtagaggtcacagattttcccttcaatacctctgaatgggagggttatctgcccaaggagagcattaggaccaaggctgggccttggggcaggtgtgctgttgtgtcgtcagcgggatctctgaagtcctcccaactaggcagagaaatcgatgatcatgacgcagtcctgaggtttaatggggcacccacagccaacttccaacaagatgtgggcacaaaaactaccattcgcctgatgaactctcagttggttaccacagagaagcgcttcctcaaagacagtttgtacaatgaaggaatcctaattgtatgggacccatctgtataccactcagatatcccaaagtggtaccagaatccggattataatttctttaacaactacaagacttatcgtaagctgcaccccaatcagcccttttacatcctcaagccccagatgccttgggagctatgggacattcttcaagaaatctccccagaagagattcagccaaaccccccatcctctgggatgcttggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggcttccggaccattcactgctag

SEQ ID NO: 2 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 3 - DNA, 1221 bp, Homo sapiens
  source 1..1221: mol_type = other DNA
atgattcacaccaacctgaagaaaaagttcagctgctgcgtcctggtctttcttctgtttgcagtcatctgtgtgtggaaggaaaagaagaaagggagttactatgattcctttaaattgcaaaccaaggaattccaggtgttaaagagtctggggaaattggccatggggtctgattcccagtctgtatcctcaagcagcacccaggacccccacaggggccgccagaccctcggcagtctcagaggcctagccaaggccaaaccagaggcctccttccaggtgtggaacaaggacagctcttccaaaaaccttatccctaggctgcaaaagatctggaagaattacctaagcatgaacaagtacaaagtgtcctacaaggggccaggaccaggcatcaagttcagtgcagaggccctgcgctgccacctccgggaccatgtgaatgtatccatggtagaggtcacagattttcccttcaatacctctgaatgggagggttatctgcccaaggagagcattaggaccaaggctgggccttggggcaggtgtgctgttgtgtcgtcagcgggatctctgaagtcctcccaactaggcagagaaatcgatgatcatgacgcagtcctgaggtttaatggggcacccacagccaacttccaacaagatgtgggcacaaaaactaccattcgcctgatgaactctcagttggttaccacagagaagcgcttcctcaaagacagtttgtacaatgaaggaatcctaattgtatgggacccatctgtataccactcagatatcccaaagtggtaccagaatccggattataatttctttaacaactacaagacttatcgtaagctgcaccccaatcagcccttttacatcctcaagccccagatgccttgggagctatgggacattcttcaagaaatctccccagaagagattcagccaaaccccccatcctctgggatgcttggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggcttccggaccattcactgctaa

SEQ ID NO: 4 - protein, 406 aa, Homo sapiens; identical to SEQ ID NO: 2

SEQ ID NO: 5 - DNA, 1212 bp, Mus musculus
  source 1..1212: mol_type = other DNA
atgattcataccaacttgaagagaaagttcagctgctttgtcctggtctttctcctgtttgccatcatctgcgtgtggaagaaagggagcgactatgaggctcttacattgcaagccaaggtattccagatgccgaagagccaggagaaagtggccgtggggcctgctccccaggctgtgttctcaaacagcaaacaagaccctaaggaaggcgttcagatcctcagttaccccagggtcacagccaaggtcaagccacagccctccttgcaggtgtgggacaaggactccacatactcaaaacttaaccccaggctgctgaagatctggaggaactatctgaacatgaataaatataaagtgtcctacaaggggccgggaccaggagtcaagttcagcgtagaggcgctgcgctgccaccttcgagaccacgtgaatgtgtctatgatagaggccacagattttcccttcaacaccactgaatgggagggttacctgcccaaggagaacttcagaaccaaggctgggccttggcataagtgtgccgtcgtgtcttctgcaggatctctgaagaactcccagctgggtcgagagattgataatcatgatgcggtcctgaggtttaatggggcacctacagacaacttccaacaggatgtgggcacaaaaactaccatccgcctagtgaactctcagttagtcaccacagaaaagcgcttcctgaaggacagtttgtacaccgaaggaatcctgattctgtgggacccatctgtgtatcatgcagacattccgcagtggtatcagaagccagactacaacttcttcgaaacctataagagttaccgaaggcttcaccccagccagcctttttacatcctcaagccccagatgccatgggaactatgggacatcattcaggaaatctctccagatctgattcagccgaatcccccatcctccggcatgctgggtatcatcattatgatgacgctgtgtgaccaagttgatatttacgagttcctcccatccaagcgcaagacagatgtgtgctactatcaccagaagttctttgacagcgcctgcacgatgggtgcctaccatccgctcctcttcgagaagaatatggtgaagcatctcaatgagggaacagatgaagacatttatttgtttgggaaagctaccctgtctggcttccggaacaatcgctgttga

SEQ ID NO: 6 - protein, 403 aa, Mus musculus
MIHTNLKRKFSCFVLVFLLFAIICVWKKGSDYEALTLQAKVFQMPKSQEKVAVGPAPQAVFSNSKQDPKEGVQILSYPRVTAKVKPQPSLQVWDKDSTYSKLNPRLLKIWRNYLNMNKYKVSYKGPGPGVKFSVEALRCHLRDHVNVSMIEATDFPFNTTEWEGYLPKENFRTKAGPWHKCAVVSSAGSLKNSQLGREIDNHDAVLRFNGAPTDNFQQDVGTKTTIRLVNSQLVTTEKRFLKDSLYTEGILILWDPSVYHADIPQWYQKPDYNFFETYKSYRRLHPSQPFYILKPQMPWELWDIIQEISPDLIQPNPPSSGMLGIIIMMTLCDQVDIYEFLPSKRKTDVCYYHQKFFDSACTMGAYHPLLFEKNMVKHLNEGTDEDIYLFGKATLSGFRNNRC

SEQ ID NO: 7 - DNA, 1212 bp, Mus musculus; identical to SEQ ID NO: 5
  source 1..1212: mol_type = other DNA

SEQ ID NO: 8 - protein, 403 aa, Mus musculus; identical to SEQ ID NO: 6

SEQ ID NO: 9 - DNA, 1212 bp, Mus musculus; identical to SEQ ID NO: 5
  source 1..1212: mol_type = other DNA

SEQ ID NO: 10 - protein, 403 aa, Mus musculus; identical to SEQ ID NO: 6

SEQ ID NO: 11 - DNA, 1212 bp, Rattus norvegicus
  source 1..1212: mol_type = other DNA
atgattcataccaacttgaagaaaaagttcagcctcttcatcctggtctttctcctgttcgcagtcatctgtgtttggaagaaagggagcgactatgaggcccttacactgcaagccaaggaattccagatgcccaagagccaggagaaagtggccatggggtctgcttcccaggttgtgttctcaaacagcaagcaagaccctaaggaagacattccaatcctcagttaccacagggtcacagccaaggtcaaaccacagccttccttccaggtgtgggacaaggactccacatactcaaaacttaaccccaggctgctgaagatctggagaaactatctgaacatgaacaaatataaagtatcctacaagggaccggggccaggagtcaagttcagcgtagaagcactgcgttgccaccttcgagaccatgtgaacgtgtctatgatagaggccacagattttcccttcaacaccactgagtgggagggttacctgcccaaggagaactttagaaccaaggttgggccttggcaaaggtgtgccgtcgtctcttctgcaggatctctgaaaaactcccagcttggtcgagagattgataatcatgatgcagttctgaggtttaatggggcccctaccgacaacttccaacaggatgtgggctcaaaaactaccattcgcctaatgaactctcagttagtcaccacagaaaagcgcttcctcaaggacagtttgtacaccgaaggaatcctaattgtatgggacccatccgtgtatcatgcagatatcccaaagtggtatcagaaaccagactacaatttcttcgaaacctataagagttaccgaaggctgaaccccagccagccattttatatcctcaagccccagatgccatgggaactgtgggacatcattcaggaaatctctgcagatctgattcagccaaatcccccatcctccggcatgctgggtatcatcatcatgatgacgctgtgtgaccaggtagatatttacgagttcctcccatccaagcgcaagacggacgtgtgctattatcaccaaaagttctttgacagcgcttgcacgatgggtgcctaccacccgctcctcttcgagaagaatatggtgaagcatctcaatgagggaacagatgaagacatttatttgtttgggaaagccaccctttctggcttccggaacattcgttgttga

SEQ ID NO: 12 - protein, 403 aa, Rattus norvegicus
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDPSVYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYHPLLFEKNMVKHLNEGTDEDIYLFGKATLSGFRNIRC

SEQ ID NO: 13 - DNA, 1197 bp, Homo sapiens
  source 1..1197: mol_type = other DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgcagcagctataggccagtcaagcggtgaactgaggactgggggtgcccgaccaccacctccactcggcgcttcatcccaaccacgtcctgggggagactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag

SEQ ID NO: 14 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 15 - DNA, 1158 bp, Homo sapiens
  source 1..1158: mol_type = other DNA
atgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag

SEQ ID NO: 16 - protein, 385 aa, Homo sapiens
MPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 17 - DNA, 1074 bp, Homo sapiens
  source 1..1074: mol_type = other DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag

SEQ ID NO: 18 - protein, 356 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFRLVFRGMSISRPNAVVGRCRMIRHSRDKKNEPNPQRFDRIAHTKETMLSDGLNSLTYQVLDVQRYPLYTQITVDIGTPS

SEQ ID NO: 19 - DNA, 678 bp, Homo sapiens
  source 1..678: mol_type = other DNA
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccagtatgaaaaaataagaagactcctgtggtga

SEQ ID NO: 20 - protein, 225 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQYEKIRRLLW

SEQ ID NO: 21 - DNA, 1227 bp, Cricetulus aureus
  source 1..1227: mol_type = other DNA
atgaggtttctgaggccggtcctgggcggcagcgccgcgatgccgggcgcgaccctgcagcgggcctgccgcctgctcgtggccgtctgcgcgctgcacctcggcgtcaccctggtctattacctctcgggccgcgacctgagccgcctgcctcagctagtcggggtttcctctacacttcggagcggcacgatcggcgccaccgccaacaagcagccccccggcgcgcggccgccgcctcctgtgggagtctcctcgaagcctcgcccgggtcccgactccagccctggtacggcttttgaccccggcctgaagagcaactggacttcggtcctagtgccccctaccactgcactgttgacgctgcccgcttgccccgaggagtccccgctgctcgttggccccatggtgattgactttaatattgctgtggacctggagcttttggcaaagaagaacccagagataaagatgggcggtcgttattcccccaaggactgtatttctcctcacaaggtggccatcattatcccatttcgcaaccggcaggagcacctcaaatattggctgtattatttgcatccggtcctacagcgccagcaactggactatggtatctacgtcatcaaccaggctggagacaccatgttcaatcgagctaagcttctcaacataggcttccaagaggccttgaaggaccatgactacaactgctttgtgttcagtgatgtggaccttattccaatggacgaccataatgcctacaggtgtttttcacagccccggcacatttctgttgcaatggacaagtttgggtttagcctgccttatgttcagtactttggaggcgtttctgccctcagtaaacaacagtttcttgccatcaatggatttcctaataattattggggctggggaggagaagatgatgacatttttaacagaatagttcataagggaatgtctatatcacgcccaaatgctgtagttggaagatgtcgcatgatacgacactcaagagacaagaaaaatgagcccaatcctcagaggtttgaccggattgctcacacaaaggaaactatgcgctttgatggtttgaactcacttacctaccaggtgttgaacgtagagagatacccattatataccaaaatcacagtggacatcgggacaccaagatagcattgtggaacaaataacagacctgaaaatggccaggggctcaga

SEQ ID NO: 22 - protein, 393 aa, Cricetulus aureus
MRFLRPVLGGSAAMPGATLQRACRLLVAVCALHLGVTLVYYLSGRDLSRLPQLVGVSSTLRSGTIGATANKQPPGARPPPPVGVSSKPRPGPDSSPGTAFDPGLKSNWTSVLVPPTTALLTLPACPEESPLLVGPMVIDFNIAVDLELLAKKNPEIKMGGRYSPKDCISPHKVAIIIPFRNRQEHLKYWLYYLHPVLQRQQLDYGIYVINQAGDTMFNRAKLLNIGFQEALKDHDYNCFVFSDVDLIPMDDHNAYRCFSQPRHISVAMDKFGFSLPYVQYFGGVSALSKQQFLAINGFPNNYWGWGGEDDDIFNRIVHKGMSISRPNAVVGRCRMIRHSRDKKNEPNPQRFDRIAHTKETMRFDGLNSLTYQVLNVERYPLYTKITVDIGTPR

SEQ ID NO: 23 - DNA, 1200 bp, Mus musculus
  source 1..1200: mol_type = other DNA
atgaggtttcgtgagcagttcctgggcggcagcgccgcgatgccgggcgcgaccctgcagcgggcctgccgcctgctcgtggccgtctgcgcgctgcacctcggcgtcaccctcgtctattacctctctggccgcgatctgagccgcctgccccagttggtcggagtctcctctacactgcagggcggcacgaacggcgccgcagccagcaagcagcccccaggagagcagcggccgcggggtgcgcggccgccgcctcctttaggcgtctccccgaagcctcgcccgggtctcgactccagccctggtgcagcttctggccccggcttgaagagcaacttgtcttcgttgccagtgcccaccaccactggactgttgtcgctgccagcttgccctgaggagtccccgctgctcgttggccccatgctgattgactttaatattgctgtggatctggagcttttggcaaagaagaacccagagataaagacgggcggccgttactcccccaaggactgtgtctctcctcacaaggtggccatcatcatcccattccgtaaccggcaggagcatctcaaatactggctgtattatttgcatcccatccttcagcgccagcaactcgactatggcatctacgtcatcaatcaggctggagacaccatgttcaatcgagctaagctgctcaatattggctttcaagaggccttgaaggactatgattacaactgctttgtgttcagtgatgtggacctcattccgatggacgaccgtaatgcctacaggtgtttttcgcagccacggcacatttctgttgcaatggacaagttcgggtttagcctgccatatgttcagtattttggaggtgtctctgctctcagtaaacaacagtttcttgccatcaatggattccctaataattattggggttggggaggagaagatgacgacatttttaacagattagttcataaaggcatgtctatatcacgtccaaatgctgtagtagggaggtgtcgaatgatccggcattcaagagacaagaaaaatgagcccaatcctcagaggtttgaccggatcgcacatacaaaggaaacgatgcgcttcgatggtttgaactcacttacctacaaggtgttggatgtacagagatacccgttatatacccaaatcacagtggacatcgggacaccgagatag

SEQ ID NO: 24 - protein, 399 aa, Mus musculus
MRFREQFLGGSAAMPGATLQRACRLLVAVCALHLGVTLVYYLSGRDLSRLPQLVGVSSTLQGGTNGAAASKQPPGEQRPRGARPPPPLGVSPKPRPGLDSSPGAASGPGLKSNLSSLPVPTTTGLLSLPACPEESPLLVGPMLIDFNIAVDLELLAKKNPEIKTGGRYSPKDCVSPHKVAIIIPFRNRQEHLKYWLYYLHPILQRQQLDYGIYVINQAGDTMFNRAKLLNIGFQEALKDYDYNCFVFSDVDLIPMDDRNAYRCFSQPRHISVAMDKFGFSLPYVQYFGGVSALSKQQFLAINGFPNNYWGWGGEDDDIFNRLVHKGMSISRPNAVVGRCRMIRHSRDKKNEPNPQRFDRIAHTKETMRFDGLNSLTYKVLDVQRYPLYTQITVDIGTPR

SEQ ID NO: 25 - DNA, 1161 bp, Mus musculus
  source 1..1161: mol_type = other DNA
atgccgggcgcgaccctgcagcgggcctgccgcctgctcgtggccgtctgcgcgctgcacctcggcgtcaccctcgtctattacctctctggccgcgatctgagccgcctgccccagttggtcggagtctcctctacactgcagggcggcacgaacggcgccgcagccagcaagcagcccccaggagagcagcggccgcggggtgcgcggccgccgcctcctttaggcgtctccccgaagcctcgcccgggtctcgactccagccctggtgcagcttctggccccggcttgaagagcaacttgtcttcgttgccagtgcccaccaccactggactgttgtcgctgccagcttgccctgaggagtccccgctgctcgttggccccatgctgattgactttaatattgctgtggatctggagcttttggcaaagaagaacccagagataaagacgggcggccgttactcccccaaggactgtgtctctcctcacaaggtggccatcatcatcccattccgtaaccggcaggagcatctcaaatactggctgtattatttgcatcccatccttcagcgccagcaactcgactatggcatctacgtcatcaatcaggctggagacaccatgttcaatcgagctaagctgctcaatattggctttcaagaggccttgaaggactatgattacaactgctttgtgttcagtgatgtggacctcattccgatggacgaccgtaatgcctacaggtgtttttcgcagccacggcacatttctgttgcaatggacaagttcgggtttagcctgccatatgttcagtattttggaggtgtctctgctctcagtaaacaacagtttcttgccatcaatggattccctaataattattggggttggggaggagaagatgacgacatttttaacagattagttcataaaggcatgtctatatcacgtccaaatgctgtagtagggaggtgtcgaatgatccggcattcaagagacaagaaaaatgagcccaatcctcagaggtttgaccggatcgcacatacaaaggaaacgatgcgcttcgatggtttgaactcacttacctacaaggtgttggatgtacagagatacccgttatatacccaaatcacagtggacatcgggacaccgagatag

SEQ ID NO: 26 - protein, 386 aa, Mus musculus
MPGATLQRACRLLVAVCALHLGVTLVYYLSGRDLSRLPQLVGVSSTLQGGTNGAAASKQPPGEQRPRGARPPPPLGVSPKPRPGLDSSPGAASGPGLKSNLSSLPVPTTTGLLSLPACPEESPLLVGPMLIDFNIAVDLELLAKKNPEIKTGGRYSPKDCVSPHKVAIIIPFRNRQEHLKYWLYYLHPILQRQQLDYGIYVINQAGDTMFNRAKLLNIGFQEALKDYDYNCFVFSDVDLIPMDDRNAYRCFSQPRHISVAMDKFGFSLPYVQYFGGVSALSKQQFLAINGFPNNYWGWGGEDDDIFNRLVHKGMSISRPNAVVGRCRMIRHSRDKKNEPNPQRFDRIAHTKETMRFDGLNSLTYKVLDVQRYPLYTQITVDIGTPR

SEQ ID NO: 27 - DNA, 1200 bp, Rattus norvegicus
  source 1..1200: mol_type = other DNA
atgaggtttcgtgagccgttcctgggcggcagcgccgcgatgccgggcgcgaccctgcagcgggcctgccgcctgctcgtggcggtctgcgcgctgcaccttggcgtcaccctggtctattacctctccggtcgcgatctgagccgcctgccccaactggtcggagtctcctcttcactgcaaggcggcacgaacggcgccgccgccagcaagcagccctcgggagagctccggccccggggcgcgcggccgccgcctcctttaggcgtctccccgaagcctcgcccgggttctgactccagccctgatgcggcttctggccccggcctgaagagcaacttgacttcggtgccaatgcccaccagcactggattgttgactctgcctgcttgccctgaggagtccccgctgctcgttggccccatggtgattgactttaatattcctgtggatctggagcttttggcaaagaagaacccagagataaagatgggcggccgttacttccccaaggactgtatctcccctcacaaggtggccatcattatcccattccgtaaccggcaggagcacctcaaatactggctgtattatttgcatccagtccttcagcgccagcaactcgactatggcatctacgtcatcaatcaggctggagacaccatgtttaatcgagctaagctgctcaacgttggctttcaagaggccttgaaagactatgactacaactgctttgtgttcagtgatgtggacctcattccaatggatgaccataatgcctacaggtgcttttcacagccacggcatatttctgtcgcaatggacaagttcgggtttagcctgccttacgttcagtattttggaggtgtctccgctctcagtaaacaacagttccttaccatcaatggatttcctaataattactggggctggggaggagaagatgatgacatttttaacagattagttcataaaggcatgtctatatcacgcccaaatgctgtggtaggcaggtgtcgcatgatccggcactcaagagacaagaaaaatgagcccaaccctcagaggtttgaccggatcgcacatacaaaggaaacgatgcgccttgatggtttgaactcacttacctaccaggtgttggacatacagagatacccgttatataccaaaatcacagtggacatcgggacaccaagatag

SEQ ID NO: 28 - protein, 399 aa, Rattus norvegicus
MRFREPFLGGSAAMPGATLQRACRLLVAVCALHLGVTLVYYLSGRDLSRLPQLVGVSSSLQGGTNGAAASKQPSGELRPRGARPPPPLGVSPKPRPGSDSSPDAASGPGLKSNLTSVPMPTSTGLLTLPACPEESPLLVGPMVIDFNIPVDLELLAKKNPEIKMGGRYFPKDCISPHKVAIIIPFRNRQEHLKYWLYYLHPVLQRQQLDYGIYVINQAGDTMFNRAKLLNVGFQEALKDYDYNCFVFSDVDLIPMDDHNAYRCFSQPRHISVAMDKFGFSLPYVQYFGGVSALSKQQFLTINGFPNNYWGWGGEDDDIFNRLVHKGMSISRPNAVVGRCRMIRHSRDKKNEPNPQRFDRIAHTKETMRLDGLNSLTYQVLDIQRYPLYTKITVDIGTPR

SEQ ID NO: 29 - protein, 449 aa, synthetic construct
  REGION 1..449: note = heavy chain of ustekinumab
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, synthetic construct
  REGION 1..214: note = light chain of ustekinumab
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC